(19) United States
(12) Patent Application Publication
(10) Pub. No.: US 2026/0072033 A1
(43) Pub. Date: Mar. 12, 2026

(54) USE OF EXTRACELLULAR VESICLES AND MICRONUCLEI OF CIRCULATING STROMAL CELLS AS PAN-CANCER BIOMARKERS FOR PREDICTING CLINICAL OUTCOMES

(71) Applicant: Creatv MicroTech, Inc., Potomac, MD (US)

(72) Inventors: Daniel L. ADAMS, Potomac, MD (US); Cha-Mei TANG, Potomac, MD (US)

(73) Assignee: Creatv MicroTech, Inc., Potomac, MD (US)

(21) Appl. No.: 19/104,551

(22) PCT Filed: Aug. 17, 2023
(86) PCT No.: PCT/US2023/030437

§ 371 Date: Feb. 18, 2025

Related U.S. Application Data
(60) Provisional application No. 63/399,060, filed on Aug. 18, 2022.
(60) Provisional application No. 63/456,588, filed on Apr. 3, 2023.
(60) Provisional application No. 63/470,595, filed on Jun. 2, 2023.
(60) Provisional application No. 63/470,536, filed on Jun. 2, 2023.

(51) Int. Cl. G01N 33/574; B01D 61/14; G01N 1/40

(52) U.S. Cl. CPC G01N 33/5759; B01D 61/147; G01N 1/4077; G01N 33/57585; B01D 2239/1216; G01N 2001/4088; G01N 2333/4725; G01N 2333/70532; G01N 2333/70553; G01N 2333/70589; G01N 2333/70596; G01N 2333/71

(57) ABSTRACT

Methods for predicting overall survival (OS) and progression free survival (PFS) of subjects having cancer, based on the presence of certain structures associated with circulating cancer associated macrophage-like cells (CAMLs), including micronuclei (MN), extracellular vesicles (EVs), enlarged polynuclearization (EPN), internalized intact cells and large internal cellular debris, are provided.

### BACKGROUND

### Field of the Invention

The present invention generally relates to the use of biomarkers in the blood and other bodily fluids to make predictions regarding overall survival and progression free survival in subjects having cancer, such as solid tumors.

### Related Art

When tumor cells break away from primary solid tumors, they penetrate into the blood or lymphatic circulation, and ultimately leave the blood stream and enter either organs or tissue to form metastasis. 90% of cancer-related deaths are caused by the metastatic process. The most common metastatic sites are the lung, liver, bone and brain. Tumor cells found in the circulation are called circulating tumor cells (CTCs). Many research publications and clinical trials show that CTCs have clinical utility in (i) providing prognostic survival and cancer recurrence information through the enumeration of CTCs in the blood stream, and (ii) providing treatment information through examination of protein expression levels, and the occurrence of gene mutations and translocations in the CTCs. However, CTCs are not consistently associated with the development and/or presence of cancer in a subject, even in stage IV cancer patients. While CTCs are found most often in stage IV of breast, prostate and colorectal cancers, they are rare in early stages of the same cancer. CTCs are also rare in other cancers.

Circulating cancer associated macrophage-like cells (CAMLs) are another cancer-related cell type that is found in the blood of subjects having cancer. CAMLs are associated with all solid tumors tested and all stages of cancer. CAMLs are polyploid and very large in size, ˜20 μm to ˜300 μm in size. These polyploid cells can be either CD45(−) or CD45(+) and express CD11c, CD14 and CD31, which confirms their origin as a myeloid lineage. They are often found in the process of engulfing CTCs and cell debris^{[14,22]}. The examination of protein expression levels in CAMLs can also assist clinicians in making informed treatment decisions.

Assays associated with the identification and characterization of biomarkers in CTCs and CAMLs, in blood and other body fluids, can be used to provide important prognostic and treatment information for a subject having cancer. The present invention is directed to providing such tools to clinicians and other important goals.

### SUMMARY

The present invention is directed prognostic methods that make use of a type of cell with unique characteristics that is found in the blood of subjects having solid tumors, including carcinoma, sarcoma, neuroblastoma and melanoma. These circulating cells, termed “circulating Cancer Associated Macrophage-like cells” (CAMLs), have been shown to be associated with the presence of solid tumors in a subject having cancer. CAMLs have been found consistently in the peripheral blood of subjects having stage I to stage IV solid tumors by microfiltration using precision microfilters.

It has been shown that an increase in the number of CAMLs and enlargement of CAMLs in the blood of a subject are both indicators for more aggressive disease with worse clinical outcomes. Thus, medical applications associated with CAMLs include, but are not limited to, use of the cells themselves as biomarkers to provide early detection of cancer and diagnosis of cancer, in particular, in the early detection and diagnosis of cancer relapse or recurrence, and in the determination of cancer mutation.

CAMLs have also been observed to possess irregular cellular characteristics not commonly found under less aggressive cancer conditions, such as (a) micronuclei (MN), (b) extracellular vesicles (EVs), (c) enlarged polynuclearization (EPN), (d) the presence of internalized intact cells and (e) large internal cellular debris. These five CAML associated structures are shown through the data presented herein as having clinical utility in making predictions regarding disease progression and patient survival which can aid in making informed treatment decisions.

More specifically, and in a first embodiment, the present invention is directed to a method for predicting overall survival (OS) and/or progression free survival (PFS) of a subject having cancer. The method comprises determining the presence of one or more of (a) micronuclei (MN), (b) extracellular vesicles (EVs), (c) enlarged polynuclearization (EPN), (d) one or more internalized intact cells, and (e) internal cellular debris in circulating cells or produced by circulating cells of a biological sample from a subject having cancer, wherein the presence of one or more of (a), (b), (c), (d) and (e) predicts lower OS and/or PFS than a subject having the same cancer without the presence of one or more of (a), (b), (c), (d) or (e).

In a second embodiment, the invention is directed to a method for predicting presence of metastatic spread and/or metastatic progression in a subject having cancer. The method comprises determining the presence of one or more of (a) MN, (b) EVs, (c) EPN, (d) internalized intact cells, and (e) internal cellular debris in circulating cells or produced by circulating cells of a biological sample from a subject having cancer, wherein the presence of one or more of (a), (b), (c), (d) and (e) predicts presence of metastatic spread and/or metastatic progression in the subject.

In a third embodiment, the invention is directed to a method for predicting cancer progression in a subject having cancer. The method comprises determining the presence of one or more of (a) MN, (b) EV, (c) EPN, (d) internalized intact cells, and (e) internal cellular debris in circulating cells or produced by circulating cells in a first biological sample and a second biological sample at a later date, and optional additional biological samples, obtained from a subject having cancer, wherein when one or more of (a), (b), (c), (d) or (e) is present in or produced by the circulating cells of the second and/or additional biological sample but not present in or produced by the circulating cells of the first biological sample, the cancer is predicted to progress in the subject, and wherein when one or more of (a), (b), (c), (d) or (e) is present in or produced by the circulating cells of the first biological sample but not present in or produced by the circulating cells of the second and/or additional biological sample, the cancer is predicted not to progress in the subject.

In a fourth embodiment, the invention is directed to a method for predicting cancer progression in a subject having cancer. The method comprises determining the presence of one or more of (a) MN, (b) EV, (c) EPN, (d) internalized intact cells, and (e) internal cellular debris in circulating cells or produced by circulating cells in a first biological sample and a second biological sample at a later date, and optional additional biological samples, obtained from a subject having cancer, wherein the first sample is obtained from the subject prior to or during cancer treatment, wherein the second sample and optional additional samples are obtained from the subject after at least one cancer treatment, wherein when one or more of (a), (b), (c), (d) or (e) is present in or produced by the circulating cells of the first biological sample but not present in or produced by the circulating cells of the second and/or additional biological sample, the cancer is predicted not to progress, and wherein when one or more of (a), (b), (c), (d) or (e) is present in the second and optional/or additional samples but not present in or produced by the circulating cells of the first biological sample, the cancer is predicted to progress.

In a fifth embodiment, the invention is directed to a method for predicting response to treatment in a subject having cancer. The method comprises determining the presence of one or more of (a) MN, (b) EV, (c) EPN, (d) internalized intact cells, and (e) internal cellular debris in circulating cells or produced by circulating cells in a first biological sample and a second biological sample at a later date, and optional additional biological samples, obtained from a subject having cancer, wherein the first sample is obtained from the subject prior to or during cancer treatment, wherein the second sample and optional additional samples are obtained from the subject after at least one cancer treatment, wherein when one or more of (a), (b), (c), (d) or (e) is present in or produced by the circulating cells of the first biological sample but not present in or produced by the circulating cells of the second and/or additional biological sample, the subject is predicted to respond to the treatment, and wherein when one or more of (a), (b), (c), (d) or (e) is present in the second and optional/or additional samples but not presence in the circulating cells of the first biological sample, the subject is predicted not to respond to the treatment.

In aspects of the first embodiment, OS and/or PFS is over a period of at least 12 months. In other aspects of the first embodiment, OS and/or PFS is over a period of at least 24 months.

The size of the biological sample in each of the embodiments of the invention is between 5 and 15 mL.

The circulating cells in each of the embodiments of the invention have the following characteristics:
(a) multiple individual nuclei and/or one or more fused nuclei having a size of about 14-64 μm;
(b) cell size of about 20-300 μm in size; and
(c) morphological shape selected from the group consisting of spindle, tadpole, round, oblong, two legs, more than two legs, thin legs, and amorphous.

The circulating cells in each of the embodiments of the invention may also have one or more, or all, of the following additional characteristics:
(d) CD14 expression;
(e) CD45 expression;
(f) EpCAM expression;
(g) vimentin expression;
(h) PD-L1 expression;
(i) monocytic and macrophage CD11c marker expression;
(j) endothelial CD146 marker expression;
(k) endothelial and macrophage CD202b marker expression;
(l) endothelial, macrophage and white blood cell CD31 marker expression; and
(m) epithelial cancer cell CK8, 18, and/or 19 marker expression.

The source of the biological sample in each of the embodiments of the invention is one or more of peripheral blood, blood, lymph node, bone marrow, cerebral spinal fluid, and urine. For example, the biological sample may be antecubital-vein blood, inferior-vena-cava blood, femoral vein blood, portal vein blood, or jugular-vein blood.

The cancer in each of the embodiments of the invention is a Stage I cancer, Stage II cancer. Stage III cancer, Stage IV cancer, carcinoma, sarcoma, neuroblastoma, melanoma, epithelial cell cancer, lung cancer, breast cancer, prostate cancer, pancreatic cancer, bladder cancer, kidney cancer, head and neck cancer, colorectal cancer, liver cancer, ovarian cancer, osteosarcoma, esophageal, brain & ONS, larynx, bronchus, oral cavity and pharynx, stomach, testis, thyroid, uterine cervix, uterine corpus cancer or other solid tumor cancers.

The circulating cells may be isolated from the biological samples for the determining steps using one or more means selected from the group consisting of size exclusion methodology, immunocapture, red blood cell lysis, white blood cell depletion, a high-molecular weight polysaccharide such as FICOLL®, electrophoresis, dielectrophoresis, flow cytometry, magnetic levitation, and various microfluidic chips, slits, channels, hydrodynamic size-based sorting, grouping, trapping, concentrating large cells, eliminating small cells, or a combination thereof.

The circulating cells may be isolated from the biological samples using size exclusion methodology that comprises using a microfilter. The microfilter may have a pore size ranging from about 5 microns to about 20 microns. Additionally, the pores of the microfilter may have a round, race-track shape, oval, square and rectangular pore shape. The microfilter may also have precision pore geometry and uniform pore distribution.

The circulating cells may be isolated using a microfluidic chip via physical size-based sorting, hydrodynamic size-based sorting, grouping, trapping, immunocapture, concentrating large cells, or eliminating small cells based on size.

The circulating cells may be isolated from the biological samples for the determining steps using a microfiltration assay.

In relevant embodiments, the treatment is one or more of chemotherapy, single drug, combination of drugs, immunotherapy, radiation therapy, chemoradiation, radiation combined with single or multiple drugs, chemoradiation combined with single or multiple drugs, cancer vaccine, and cell therapy. As a non-limiting example, the treatment may be a cancer vaccine and the subject expresses at least one HLA allele.

In relevant embodiments, the subject may be treated with one or more of chemotherapy, single drug, combination of drugs, immunotherapy, radiation therapy, chemoradiation, radiation combined with single or multiple drugs, chemoradiation combined with single or multiple drugs, cancer vaccine, and cell therapy. As a non-limiting example, the immunotherapy may be PD-L1 immunotherapy.

CAMLs and the noted CAML associated structures may be used independently as cancer markers, or in combination with biomarker expression by other circulating cells, such as circulating tumor cells (CTCs). Suitable CTC subtypes include, but are not limited to, pathologically definable CTCs (PDCTCs), apoptotic CTCs, and the CTC subtype undergoing epithelial mesenchymal transition cells (EMTCTCs). Other suitable circulating cell types include circulating cancer associated vascular endothelial cells (CAVEs). The presence of these additional cell types themselves and also cell-free DNA (cfDNA), circulating tumor DNA (ctDNA), methylated DNA, proteomic, metabolomic, lipidomic and other biomarkers may provide a more complete understanding of a patient's disease.

In each aspect and embodiment of the invention, the methods further comprise administering a therapeutically effective amount of a cancer treatment to the subject. The subject may be a subject in which the OS and PFS is predicted to be shorter than the OS and PFS of another subject.

### BRIEF DESCRIPTION OF THE DRAWINGS

FIG. 1 shows cell differentiation markers used to identify and subtype CAMLs.

FIG. 2 shows examples of three CAMLs with micronuclei (MN). White dashed squares encircle the MN. Enlargement and recoloring of the white dashed squares enable better observation of the MN within the cells.

FIG. 3A shows an image of a CAML with micronuclei and enlarged nucleus. A single CAML with enlarged nucleus (a, green-blue), CD45 staining (b, violet), diffuse cytokeratin (c, green) and DAPI (d, blue) indicating cell structure (box=85 μm). Enlarged portion of the cell with one micronuclei budding from the primary nucleic mass (e, white arrow) and two separate distinct micronuclei also within the cell cytoplasm (box=20 μm).

FIG. 3B shows a micronuclei positive CAML (a. 66 μm diameter) stained with PD-L1 (red) and DAPI (blue). MN size varies from 4 μm (b.) to 2 μm (c.).

FIG. 4 shows three subtypes of EVs, separated based on method of formation and size exosomes (˜30 nm-100 nm), microvesicles (˜100 nm-1 μm) and apoptotic bodies (˜1-5 μm). EVs originate from CAMLs which leave the tumor site and enter circulation.

FIG. 5 shows progression free survival (PFS) of stromal cells based on the presence or absence of micronuclei (MN) over 24 months.

FIG. 6 shows overall survival (OS) of stromal cells based on the presence or absence of micronuclei (MN) over 24 months.

FIG. 7 shows average PD-L1 expression for all MN+ cells (n=185) was 338 while average expression in MN-cells (n=347) was 253. Median (red line), high error bar (max), low error bar (min).

FIG. 8 shows Kaplan-Meyer of progression free survival (PFS) and overall survival (OS) for micronuclei positivity in colorectal patients at first blood draw. Panel a. Kaplan-Meyer of PFS for patients who presented positivity for micronuclei (red) or negative (blue). Panel b. Kaplan-Meyer of OS for patients who presented positivity for micronuclei (red) or negative (blue).

FIG. 9 shows Kaplan-Meier of PFS and OS for Micronuclei positivity in patients at first blood draw. Panel a. Kaplan-Meier of PFS for patients who presented positivity for micronuclei (blue) or negative (red). Panel b. Kaplan-Meier of OS for patients who presented positivity for micronuclei (blue) or negative (red).

FIG. 10 shows single factor ANOVA comparing PD-L1 expression in cells with MN or without MN.

FIG. 11 shows T-tests comparing PD-L1 expression in cells without MN, 1 MN, 2 MN or ≥3 MN.

FIG. 12 shows micronuclei presence in the various clinical parameters. Panel a. Linear regression plot of the relationship of CAML number versus micronuclei number in each patient at baseline sampling. Panel b. Micronuclei presence based on patients currently on chemotherapy (i.e. genotoxin) or patients that were newly diagnosed and treatment naive. Panel c. MN presence based on non-metastatic or metastatic spread. Graph d. Micronuclei frequency in all CAMLs (n=776). 91.0% of CAMLs (blue) were negative for micronuclei, while only 9% were positive for at least 1 micronuclei.

FIG. 13 shows case studies tracking average micronuclei in subsequent blood draws. Panel a. is Patient A. After progression on maintenance therapy (Xeloda, red box), FOLFOX (orange box) was started. MN were found to increase after 4 therapy cycles which correlated to an increase in tumor (+17%). Therapy was halted due to an infection. MN then increased further correlating with a finding of new lesions at cycle 6. FOLFOX was then restarted, and MN continued to increase, the patient then dropped from study. Panel b. is Patient B. A patient with progressive disease on FOLFOX was started on a single agent CCR5 inhibitor (Leronlimab, blue box), which was followed by a decrease in MN correlating with a reduction (−39%) in tumor size. MNs then increased slightly, which correlated with a slight increase in tumor size (+11%) at which point FOLFIRI (purple box) was added to leronlimab. After FOLFIRI induction, a drop in MN was seen which correlated with stable disease.

FIG. 14 shows Kaplan-Meier of PFS and OS for EV positivity in CAMLs in metastatic non-small cell lung carcinoma (mNSCLC) patients. Top panel is Kaplan-Meier of PFS for patients who presented positivity for EVs (red) or negative (black). Bottom panel is Kaplan-Meier of OS for patients who presented positivity for EVs (red) or negative (black).

FIG. 15 shows an EV positive CAML (45 μm in diameter) stained for PD-L1. Multiple PD-L1 positive EVs ranging in size from <1 μm to 3 μm.

FIG. 16 shows Kaplan-Meier of PFS and OS for EV positivity in CAMLs in patients at first blood draw from 130 patients with an array of cancer types. Panel a. Kaplan-Meier of PFS for patients who presented positivity for EVs (blue) or negative (red). Panel b. Kaplan-Meier of OS for patients who presented positivity for EVs (blue) or negative (red).

FIG. 17 shows tracking of changes in EV presence in response to CRT in a single patient over 15 months. Patient received chemoradiation therapy (CRT) at baseline (BL) and EV presence dropped 33% after CRT completion seen at the T1 blood draw. EV presence increases from T2 to T5 at which point the patient was found to have disease progression is confirmed by radiographic imaging (PET/CT). The patient was started on Ipilumab and was then found to progressed with additional metastases at T7.

FIG. 18 shows Kaplan-Meier of PFS and OS for EV positivity in CAMLs in metastatic non-small cell lung carcinoma (mNSCLC) patients. Panel a. Kaplan-Meier of PFS for patients who presented positivity for EVs (red) or negative (blue). Panel b. is Kaplan-Meier of OS for patients who presented positivity for EVs (red) or negative (blue).

FIG. 19 shows Kaplan-Meier of PFS and OS for EV positivity in CAMLs in metastatic non-small cell lung carcinoma (mNSCLC) patients. Treated with or without immunotherapy (IMT).

FIG. 20 shows Kaplan-Meier of PFS and OS for Enlarged DAPI nuclei in CAMLs in patients at first blood draw. Panel a. Kaplan-Meier of PFS for patients who presented with nuclei>465 μm² (red) or <465 μm² (blue). Panel b. Kaplan-Meier of OS for patients who with nuclei>460 μm² (red) or <465 μm² (blue).

FIG. 21 shows Kaplan-Meier of PFS and OS for WBC CAML positivity in patients at first blood draw. Panel a. Kaplan-Meier of PFS for patients who presented positivity for CAML WBCs (blue) or negative (red). Panel b. Kaplan-Meier of OS for patients who presented positivity for CAML WBCs (blue) or negative (red).

FIG. 22 shows Kaplan-Meier of PFS and OS for Debris positivity in patients at first blood draw. Panel a. Kaplan-Meier of PFS for patients who presented positivity for debris (blue) or negative (red). Panel b. Kaplan-Meier of OS for patients who presented positivity for debris (blue) or negative (red).

FIG. 23 shows Kaplan-Meier of PFS and OS for CAMLs with MN, EV and/or a >465 μm² nuclei in patients at first blood draw. Panel a. Kaplan-Meier of PFS for patients who presented positivity for CAMLs with MN, EV or >465 μm² nuclei (blue) or negative (red). Panel b. Kaplan-Meier of OS for patients who presented positivity for CAMLs with MN, EV or >465 μm² nuclei (blue) or negative (red).

### DETAILED DESCRIPTION

The matters defined in the description such as a detailed construction and elements are nothing but the ones provided to assist in a comprehensive understanding of the invention. Accordingly, those of ordinary skill in the art will recognize that various changes and modifications of the embodiments described herein can be made without departing from the scope and spirit of the invention.

Cancer is one of the most feared illness in the world, affecting all populations and ethnicities in all countries. Approximately 40% of both men and women will develop cancer in their lifetime. In the United States alone, at any given time there are more than 12 million cancer patients, with 1.7 million new cancer cases and more than 0.6 million deaths estimated yearly. Cancer death worldwide is estimated to be about 8 million annually, of which 3 million occur in developed countries where patients have access to treatment.

Liquid biopsies provide real-time, sequential tracking of diagnostically-important circulating cells isolated from a subject having cancer. Cells such as circulating tumor cells (CTCs) are found in the peripheral blood of cancer patients and previous work has shown that CTC-based assays can be used as a substitute to tissue biopsies^{[1-4]}.

Recently, another circulating cell associated with cancer has been identified in the peripheral blood of cancer patients. This cancer stromal cell subtype has been termed a cancer associated macrophage-like cell or CAML. CAMLs have been identified in the blood using a non-affinity microfiltration based method which captures both CTCs and CAMLs, and allows for singular or parallel analysis of these cancer specific circulating cell subtypes^{[1, 6-16]}. CAMLs are a recently defined circulating myeloid derived stromal cell, found in all the stages of invasive malignancy and in various solid malignancies (e.g. breast, prostate, non-small cell lung carcinoma (NSCLC), and pancreatic)^{[11, 13, 14, 17]}. CAMLs are specialized myeloid polyploid cells in the blood in all stages of solid tumors. They are easy to identify by their large size (greater than 20 μm), polyploid nucleus and morphologies: round, rod shaped, with one tail or two tails 180 degrees apart. CAMLs typically express CD31, CD14, CD45 and cytokeratin, and can also express EpCAM, CD146, CD11c and tie2^{[11, 13, 14, 17]}.

While the simple presence of one or more of these circulating cell types in a blood sample obtained from a subject having cancer provides diagnostic information, the presence of selected structures or biomarkers in the cells can provide additional information. For example, and as discussed herein, the present invention is based on the discovery by the inventors that CAMLs possess irregular cellular characteristics not commonly found under less aggressive cancer conditions, such as (a) micronuclei (MN), (b) extracellular vesicles (EVs), (c) enlarged polynuclearization (EPN), (d) the presence of internalized intact cells and (e) large internal cellular debris. These five CAML associated structures are shown through the data presented herein as having clinical utility in making predictions regarding disease progression and patient survival which can aid in making informed treatment decisions. A detailed discussion regarding these CAML associated structures is provided after the following details regarding relevant cell types.

### Circulating Cancer Associated Macrophage-Like Cells (CAMLs)

As defined herein, the circulating cells used in the methods of the invention can be termed “CAMLs”. Each reference to “circulating cells” is synonymous with CAMLs, and each reference to “CAMLs” is synonymous with circulating cells. Whether termed CAMLs or “circulating cells” these cells are characterized by having one or more of the following features:
CAMLs have a large, atypical polyploid nucleus or multiple individual nuclei, often scattered in the cell, though enlarged fused nucleoli are common. CAML nuclei generally range in size from about 10 μm to about 70 μm in diameter, more commonly from about 14 μm to about 64 μm in diameter.
For many cancers, CAMLs express the cancer marker of the disease. For example, CAMLs associated with epithelial cancers may express CK 8, 18 or 19, EpCAM, vimentin, etc. The markers are typically diffused, or associated with vacuoles and/or ingested material. The staining pattern for any marker is nearly uniformly diffused throughout the whole cell. For sarcomas, neuroblastomas and melanomas, other markers associated with the cancers can be used instead of CK 8, 18, 19.
CAMLs can be CD45 positive or CD45 negative, and the present invention encompasses the use of both types of CAMLs.
CAMLs are large, approximately 20 micron to approximately 300 micron in size by the longest dimension.
CAMLs are found in many distinct morphological shapes, including spindle, tadpole, round, oblong, two legs, more than two legs, thin legs, or amorphous shapes^{1,2}.
CAMLs from carcinomas typically have diffused cytokeratins.
If CAMLs express EpCAM, EpCAM is typically diffused throughout the cell, or associated with vacuoles and/or ingested material, and nearly uniform throughout the whole cell, but not all CAML express EpCAM, because some tumors express very low or no EpCAM.
If CAMLs express a marker, the marker is often diffused throughout the cell, or associated with vacuoles and/or ingested material, and nearly uniform throughout the whole cell, but not all CAML express the same markers with equal intensity and for a limited number of markers, the markers are not distributed equally throughout the cell.
CAMLs often express markers associated with the markers of the tumor origin; e.g., if the tumor is of prostate cancer origin and expresses PSMA, then CAMLs from such a patient also expresses PSMA. As another example, if the primary tumor is of pancreatic origin and expresses PDX-1, then CAMLs from such a patient also expresses PDX-1. As further example, if the primary tumor or CTC of the cancer origin express CXCR-4, then CAMLs from such a patient also express CXCR-4.
If the primary tumor or CTC originating from the cancer expresses a biomarker of a drug target, CAMLs from such a patient also express the biomarker of the drug target. An example of such a biomarker of immunotherapy is PD-L1.
CAMLs express monocytic markers (e.g. CD11c, CD14) and endothelial markers (e.g. CD146, CD202b, CD31).
CAMLs have the ability to bind Fc fragments.

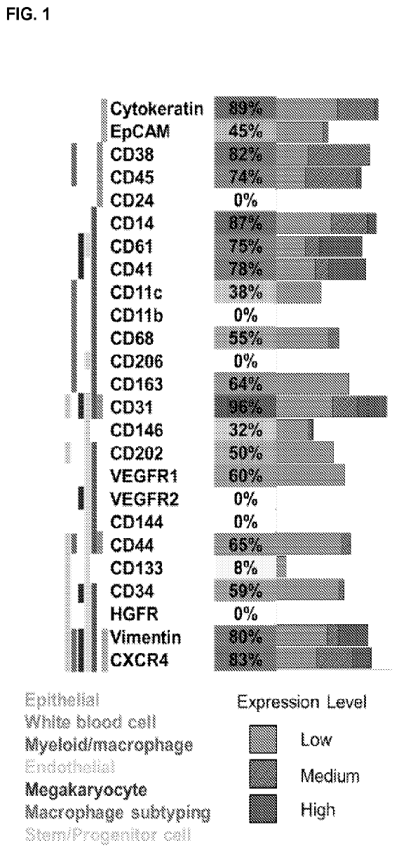

An extensive set of markers were evaluated for their expression on CAMLs, and the results are shown in FIG. 1. In one aspect of the invention, the CAMLs of the present invention express 1, 2, 3, 4, 5, 6, 7, 8, 9, 10, 11, 12, 13, 14, 15, 16, 17, 18, 19, 20 or all 21 of the markers shown in FIG. 1. The markers were screened against 1118 CAMLs from 93 different patients with different cancers. CAMLs were initially isolated and identified with DAPI, cytokeratin, and CD45; then sequentially restrained with total of 27 markers including myeloid/macrophage, white blood cell, megakaryocyte, epithelial, endothelial, progenitor/stem, and motility markers. As can be seen from FIG. 1, marker expression ranges from 0% to 96%. Almost all CAMLs were found to express levels of CD31, and commonly co-expressed cytokeratin, CD14, CXCR4, vimentin and other markers. However, while CAMLs contained clear myeloid lineage marker (CD14), CD31 marker was expressed more often at 96%.

CAMLs also present with numerous phenotypes which do not appear to match the understanding of classical cellular differentiation (i.e. co-expression of CD45 [leukocyte] and cytokeratin [epithelial], CD11c/CD14 [macrophage] and CD41 [macrophage/megakaryocyte], CD146 [endothelial] and CD61 [macrophage/endothelial/megakaryocyte], CD31 [white blood cell/macrophage/endothelial/megakaryocyte/stem cell] and CD68/CD163 [macrophage]). Many of the markers appear on multiple cell types. Combined, these data show CAMLs are myeloid-derived cells early in their differentiation process that possess many phenotypic attributes associated with stem cell and proangiogenic capabilities.

CAMLs can be visualized by colorimetric stains, such as H&E, or fluorescent staining of specific markers as shown in FIG. 1. For the cytoplasm, CD31 is the most positive phenotype. CD31 alone, or in combination with other positive markers in FIG. 1, or cancer markers associated with the tumor are recommended.

Thus, and in the various embodiments and aspects of the invention, the circulating cells (CAMLs) can be defined as having each of the following characteristics:
(a) multiple individual nuclei and/or one or more fused nuclei having a size of about 14-64 μm;
(b) cell size of about 20-300 μm in size; and
(c) morphological shape selected from the group consisting of spindle, tadpole, round, oblong, two legs, more than two legs, thin legs, and amorphous.

In certain aspects of the embodiments of the invention, the circulating cells (CAMLs) can be further identified as having one or more of the following additional characteristics:
(d) CD14 expression;
(e) CD45 expression;
(f) EpCAM expression;
(g) vimentin expression;
(h) PD-L1 expression;
(i) monocytic and macrophage CD11c marker expression;
(j) endothelial CD146 marker expression;
(k) endothelial and macrophage CD202b marker expression;
(l) endothelial, macrophage and white blood cell CD31 marker expression; and
(m) epithelial cancer cell CK8, 18, and/or 19 marker expression.

Currently, there is no officially designated term for CAMLs in the literature. Other terminology that appears in the literature and describes the same cell type includes the following:
circulating stromal cells,
immune stromal cells,
circulating cancer-associated cells,
circulating tumor-macrophage hybrid cells,
cell-cell fusion cells,
tumor-associated macrophage hybrid cells,
dual-positive circulating cells (referencing the presence of CK and CD45 markers),
fusion of tumor cells with macrophages,
circulating giant tumor-macrophage fusion cells,
tumor cell-macrophage fusion cells,
fusion hybrid cells,
circulating hybrid cells, and
tumor hybrid cells.

### Circulating Tumor Cells

As defined herein, CTCs associated with carcinomas express a number of cytokeratins (CKs). CK 8, 18, & 19 are the cytokeratins most commonly expressed and used in diagnostics, but surveying need not be limited to these markers alone. The surface of solid tumor CTCs usually express epithelial cell adhesion molecule (EpCAM). However, this expression is not uniform or consistent. CTCs do not express any CD45 because it is a white blood cell marker. In assays to identify tumor-associated cells, such as CTCs and CAMLs, it is sufficient to use antibodies against markers associated with the solid tumor such as CK 8, 18 and 19, or antibodies against CD45 or DAPI.

Different subgroups of CTCs upregulate and/or down regulate phenotypes and marker expression in relation to tumor progression, tumor spread, and in response to tumor treatments. Therefore, assessing CTCs and the markers expressed by such cells in the peripheral blood can provide important information regarding the status of the cancer in the subject.

CTCs that can be used as cancer diagnostics, as well as means for screening and monitoring treatment and determining the susceptibility of a tumor in a particular subject to a particular treatment, can be divided into three subgroups. The first subgroup is pathologically definable CTCs (PDCTCs)^{[6-10]}. PDCTCs can be characterized by: a “cancer-like” nucleus stained by DAPI; cytokeratin having a filamentous pattern; expression of one or more of CK 8, 18 and 19 (CTCs from epithelial cancers usually express at least CK 8, 18 and 19); lack of CD45 expression.

The second subgroup is apoptotic CTCs. When a CTC dies, the cytokeratin pattern degrades into dots. Therefore, early apoptotic CTC have some cytokeratin dots and later apoptotic CTCs have all of the cellular cytokeratin degraded into dots. Apoptotic CTCs can also be characterized as expressing CK 8, 18 and 19; a degrading nuclei; lack of CD45 expression.

In the third subgroup of CTCs, the cells are undergoing epithelial to mesenchymal transition (EMTCTCs)^{[2, 3, 5-9]}. EMT is a gradual morphogenetic process, and EMTCTCs encompass cells in various stages of transition^{[6]}. EMTCTCs can be generally described by the down regulation of epithelial proteins, e.g. EpCAM and CK, and the upregulation of mesenchymal stem cell proteins, e.g. vimentin and CD34^{[13]}. EMTCTC subtyping is typically performed using non-proteomic methods, i.e. mRNA expression or DNA analysis^{[13]}.

A further type of circulating cell associated with cancer that may serve as a diagnostic is the cancer-associated vascular endothelial cell or CAVE. CAVEs are a subtype of circulating endothelial cells. Tumors require blood supply provided by tumor endothelial cells. CAVES are tumor endothelial cells that have broken off from the tumor site into the blood stream. CAVEs are often found in clusters. CAVEs express cytokeratin and various subtypes endothelial cell markers such as CD31, CD146, CD144, CD105, but do not express CD14 or CD45^{[20]}.

Combining staining techniques with morphology, pathologically-definable CTCs (PDCTC), apoptotic CTCs and CAMLs can be identified 161.

### Predictive Methods

As suggested above, unique characteristics of CAMLs make them well-suited for use in clinical methodology including methods of screening and diagnosis diseases such as cancer, monitoring treatment, monitoring of disease progression and recurrence.

It has been shown that an increase in the number of CAMLs and enlargement of CAMLs in the blood of a subject are both indicators for more aggressive disease with worse clinical outcomes. However, CAMLs have also been observed to possess other irregular cellular characteristics not commonly found under less aggressive cancer conditions, such as (a) micronuclei (MN), (b) formation of extracellular vesicles (EVs), (c) enlarged polynuclearization (EPN), (d) the presence of internalized intact cells and (e) large internal cellular debris. These five “CAML associated structures” form the basis of the present invention.

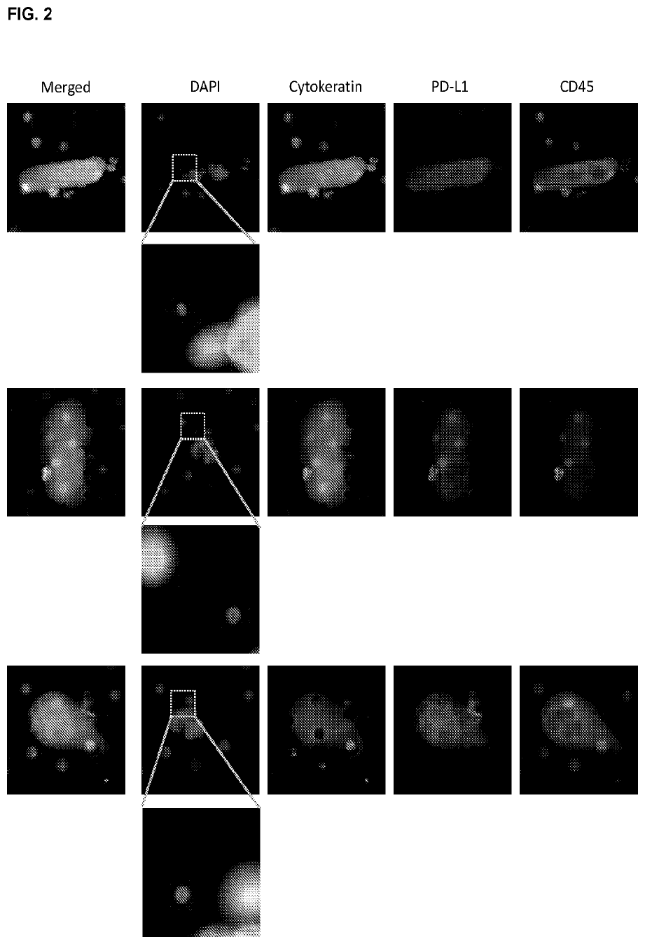

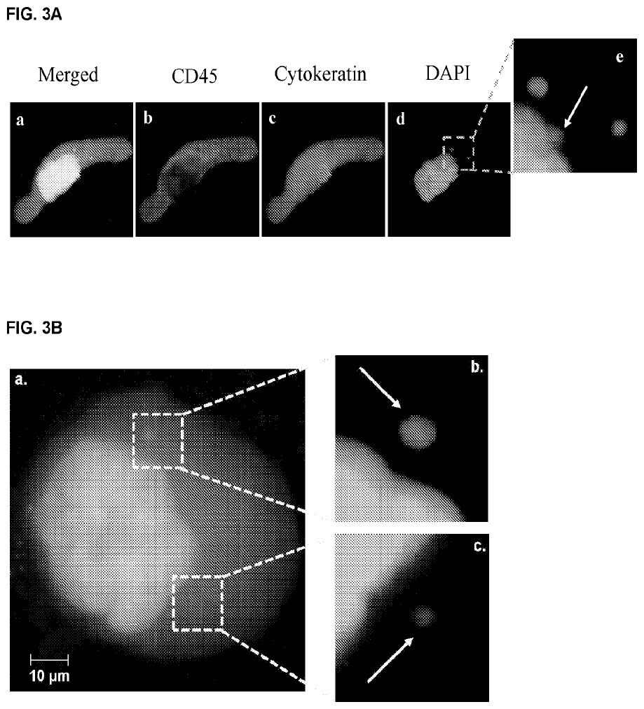

Micronuclei (MN) are a result of biological DNA repair mechanisms^{33,34}, forming due to internal chromosomal aberrations which indicate sub-clonal cancer populations with higher cell survivability and drug therapy resistance. MN are often observed as small fragments of nucleic acids excised from a primary nucleus in both tumor cells and surrounding tumor immune stromal cells (FIGS. 2 and 3). Cells with damaged DNA undergoing repair mechanisms, such as those that form MN, appear to have upregulated expression of programmed cell death ligand (PD-L1). MN are defined as small DAPI+ circular formations within the cytoplasm of a cell, separate from the primary nucleus. MN vary in size between 10 nm and 10 μm, but are typically between 0.5 and 4 μm.

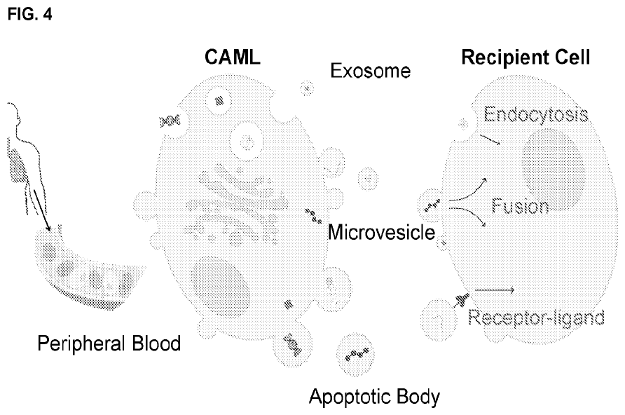

Extracellular vesicles (EVs), which include exosomes, microvesicles, and apoptotic bodies, are involved in cellular communication, tumor growth, and metastasis in cancer (FIG. 4). EVs are broadly defined as membrane bound vesicles secreted by cells into the extracellular space. EVs are important players in cellular communication and cancer recognition, intricately involved in tumor growth, progression, and metastasis in cancer. EV budding can be observed from cells, such as CAMLs, as small bulbous protrusions from the cell periphery. CAML EV budding is characterized as small bulbous protrusions vary in size between 1 and 10 μm, but are typically between 0.5 and 5 μm.

Enlarged polynuclearization (EPN), or an abnormally high quantity of nucleic acids in a cell, is an indicator of abnormal cell function that can result from many diseased states including viral infection or cancer. As used herein, EPNs are nuclear masses having an area of >450 μm². They may also be defined as having a nuclear mass area of >460 μm², >1000 μm², >2000 μm² or >3000 μm².

Internalized intact cells or the presence of large internal cell debris can result from a number of biological mechanisms including phagocytosis, internal cellular repair and emperipolesis, and may result in the down regulation of an immunological response. As used herein, internalized intact cells refers to whole or partial white blood cells (WBCs), whole or partial CTCs, or WBCs bound to CAMLs. As used herein, large internal cell debris internalized, non-disintegrated cellular debris or other internal structures within CAMLs.

Identifying any of these features can be difficult within tumor biopsies and are largely understudied in context of liquid biopsies in cancer research. However, as reported herein each of these five observable biological features was assayed in CAMLs from a variety of cancer patients to evaluate their use as clinical predictors, including their association with more aggressive diseases that are likely to have worse clinical outcomes as determined by faster progression, higher mortality rates and/or having increased risk of metastatic relapse. MN, EVs, EPN, internalized intact cells, and internal cellular debris were all observed in CAMLs in numerous stages of cancer and in an array of different solid tumor subtypes.

As defined there, these observations have been translated into the present invention.

As suggested in the Summary above, the present invention is directed in a first embodiment to methods for predicting overall survival (OS) and/or progression free survival (PFS) of a subject having cancer. The methods comprise determining the presence of one or more of (a) micronuclei (MN), (b) extracellular vesicles (EVs), (c) enlarged polynuclearization (EPN), (d) one or more internalized intact cells, and (e) internal cellular debris in circulating cells or produced by circulating cells of a biological sample from a subject having cancer, wherein the presence of one or more of (a), (b), (c), (d) and (e) predicts lower OS and/or PFS than a subject having the same cancer without the presence of one or more of (a), (b), (c), (d) or (e).

In a second embodiment, the invention is directed to methods for predicting presence of metastatic spread and/or metastatic progression in a subject having cancer. The methods comprise determining the presence of one or more of (a) MN, (b) EVs, (c) EPN, (d) internalized intact cells, and (e) internal cellular debris in circulating cells or produced by circulating cells of a biological sample from a subject having cancer, wherein the presence of one or more of (a), (b), (c), (d) and (e) predicts presence of metastatic spread and/or metastatic progression in the subject.

In a third embodiment, the invention is directed to methods for predicting cancer progression in a subject having cancer. The methods comprise determining the presence of one or more of (a) MN, (b) EV, (c) EPN, (d) internalized intact cells, and (e) internal cellular debris in circulating cells or produced by circulating cells in a first biological sample and a second biological sample at a later date, and optional additional biological samples, obtained from a subject having cancer, wherein when one or more of (a), (b), (c), (d) or (e) is present in or produced by the circulating cells of the second and/or additional biological sample but not present in or produced by the circulating cells of the first biological sample, the cancer is predicted to progress in the subject, and wherein when one or more of (a), (b), (c), (d) or (e) is present in or produced by the circulating cells of the first biological sample but not present in or produced by the circulating cells of the second and/or additional biological sample, the cancer is predicted not to progress in the subject.

In a fourth embodiment, the invention is directed to methods for predicting cancer progression in a subject having cancer. The methods comprise determining the presence of one or more of (a) MN, (b) EV, (c) EPN, (d) internalized intact cells, and (e) internal cellular debris in circulating cells or produced by circulating cells in a first biological sample and a second biological sample at a later date, and optional additional biological samples, obtained from a subject having cancer, wherein the first sample is obtained from the subject prior to or during cancer treatment, wherein the second sample and optional additional samples are obtained from the subject after at least one cancer treatment, wherein when one or more of (a), (b), (c), (d) or (e) is present in or produced by the circulating cells of the first biological sample but not present in or produced by the circulating cells of the second and/or additional biological sample, the cancer is predicted not to progress, and wherein when one or more of (a), (b), (c), (d) or (e) is present in the second and optional/or additional samples but not present in or produced by the circulating cells of the first biological sample, the cancer is predicted to progress.

In a fifth embodiment, the invention is directed to methods for predicting response to treatment in a subject having cancer. The methods comprise determining the presence of one or more of (a) MN, (b) EV, (c) EPN, (d) internalized intact cells, and (e) internal cellular debris in circulating cells or produced by circulating cells in a first biological sample and a second biological sample at a later date, and optional additional biological samples, obtained from a subject having cancer, wherein the first sample is obtained from the subject prior to or during cancer treatment, wherein the second sample and optional additional samples are obtained from the subject after at least one cancer treatment, wherein when one or more of (a), (b), (c), (d) or (e) is present in or produced by the circulating cells of the first biological sample but not present in or produced by the circulating cells of the second and/or additional biological sample, the subject is predicted to respond to the treatment, and wherein when one or more of (a), (b), (c), (d) or (e) is present in the second and optional/or additional samples but not presence in the circulating cells of the first biological sample, the subject is predicted not to respond to the treatment.

The circulating cells in each of the embodiments of the invention have the following characteristics:
(a) multiple individual nuclei and/or one or more fused nuclei having a size of about 14-64 μm;
(b) cell size of about 20-300 μm in size; and
(c) morphological shape selected from the group consisting of spindle, tadpole, round, oblong, two legs, more than two legs, thin legs, and amorphous.

The circulating cells in each of the embodiments of the invention may also have one or more, or all, of the following additional characteristics:
(d) CD14 expression;
(e) CD45 expression;
(f) EpCAM expression;
(g) vimentin expression;
(h) PD-L1 expression;
(i) monocytic and macrophage CD11c marker expression;
(j) endothelial CD146 marker expression;
(k) endothelial and macrophage CD202b marker expression;
(l) endothelial, macrophage and white blood cell CD31 marker expression; and
(m) epithelial cancer cell CK8, 18, and/or 19 marker expression.

In each of the methods of the invention, the number of circulating cells in which the CAML associated structures are evaluated can range from 1 to >100 cells, and includes 1 cell, and the ranges of 1-2 cells, 1-3 cells, 1-4 cells, 1-5 cells, 1-6 cells, 1-7 cells, 1-8 cells, 1-9 cells, 1-10 cells, 1-20 cells, 1-30 cells, 1-40 cells, 1-50 cells, 1-100 cells and 1-200 cells.

As used herein, overall survival (OS) means the length of time survived by a subject having cancer from a selected date, such as the date of diagnosis, the date on which treatment began, and the date on which blood is drawn to assess cancer progression.

As used herein, progression free survival (PFS) means the length of time survived by a subject having cancer from a selected date, such as the date on which treatment began or the date on which blood is drawn to assess cancer progression, and where the cancer has not worsened or progressed.

In each of the methods of the invention, OS or PFS, or both, is over a period of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 months, or more. In one aspect of the invention, OS or PFS, or both, is over a period of at least about 12 months or at least about 24 months.

In each of the methods of the invention, when OS and/or PFS is predicted to be “shortened” or “worse”, OS and/or PFS is shorter in duration then would be the case for a subject having cancer in which the CAML associated structures had not been found.

In each of the methods of the invention, it will be apparent that the amount of the biological sample in which the circulating cells (e.g., CAMLs) are assayed can vary. However, the biological sample should generally be at least about 2.5 mL. The amount of biological sample may also be at least about 3, 4, 5, 6, 7, 7.5, 8, 9, 10, 11, 12, 12.5, 13, 14, 15, 16, 17, 17.5, 18, 19, 20, 21, 22, 22.5, 23, 24, 25, 26, 27, 27.5, 28, 29, or 30 mL, or more. The amount of biological sample may also be between about 2.5 and 20 mL, between about 5 and 15 mL, or between about 5 and 10 mL. In one aspect of the invention, the biological sample is about 7.5 mL.

In each of the embodiments and aspects of the invention, the source of the biological sample may be, but is not limited to, one or more of peripheral blood, blood, lymph node, bone marrow, cerebral spinal fluid, and urine. When the biological sample is blood, the blood may be antecubital-vein blood, inferior-vena-cava blood femoral vein blood, portal vein blood, or jugular-vein blood, for example. The sample may be a fresh sample or a cryo-preserved sample that is thawed^{[14].}

In each of the embodiments and aspects of the invention, the cancer may be a Stage I cancer, Stage II cancer, Stage III cancer, Stage IV cancer, carcinoma, sarcoma, neuroblastoma, melanoma, epithelial cell cancer, lung cancer, breast cancer, prostate cancer, pancreatic cancer, bladder cancer, kidney cancer, head and neck cancer, colorectal cancer, liver cancer, ovarian cancer, osteosarcoma, esophageal, brain & ONS, larynx, bronchus, oral cavity and pharynx, stomach, testis, thyroid, uterine cervix, uterine corpus cancer or other solid tumor cancers. The skilled artisan will appreciate that the methods of the invention are not limited to particular forms or types of cancer and that they may be practiced in association with a wide variety of cancers

In each of the embodiments and aspects of the invention, the circulating cells (e.g., CAMLs) are isolated from the biological samples for the determining steps using one or more means selected from size exclusion methodology, immunocapture, red blood cell lysis, white blood cell depletion, a high-molecular weight polysaccharide such as FICOLL@, electrophoresis, dielectrophoresis, flow cytometry, magnetic levitation, and various microfluidic chips, slits, channels, hydrodynamic size-based sorting, grouping, trapping, concentrating large cells, eliminating small cells, or a combination thereof. In a particular aspect, the size exclusion methodology comprises use of a microfilter.

In one aspect of the invention, circulating cells are isolated from the biological samples using size exclusion methodology that comprises using a microfilter. Suitable microfilters can have a variety of pore sizes and shapes. The microfilter may have a pore size ranging from about 5 microns to about 20 microns. In certain aspects of the invention, the pore size is between about 5 and 10 microns; in other aspects, the pore size is between about 7 and 8 microns. The larger pore sizes will eliminate most of the WBC contamination on the filter. The pores of the microfilter may have any shape, with acceptable shapes including round, race-track shape, oval, slit, square, rectangular and/or other shapes. The microfilter may have precision pore geometry, uniform pore distribution, more than one pore geometry, and/or non-uniform distribution. The microfilter may be single layer, or multi-layers with different shapes on different layers.

In another aspect of the invention, circulating cells are isolated from the biological samples using a microfluidic chip via physical size-based sorting, slits, channels, hydrodynamic size-based sorting, grouping, trapping, immunocapture, concentrating large cells, or eliminating small cells based on size. The circulating cell capture efficiency can vary depending on the collection method. The size of a circulating cell that can be captured on different platforms can also vary depending, for example, on the identity of the cell. Collection of circulating cells using CELLSIEVE™ microfilters provides 100% capture efficiency and high quality cells.

In another aspect of the invention, when the biological sample is peripheral blood or blood, the sample may be collected from a subject using a blood collection tube. CELLSAVE™ blood collection tubes (Menarini Silicon Biosystems Inc., San Diego, CA), for example, provide stable cell morphology and size.

In another aspect of the invention, circulating cells can be captured and analyzed without specifically identifying the cells as CAML cells per se. Instead, one may simply identify the cells based on size of the cytoplasm and nucleus. Examples for doing so are techniques using color metric stains, such as H&E stains, or merely looking at CK (+) cells.

In a further aspect of the invention, circulating cells are isolated from the biological samples using a CELLSIEVE™ microfilter low-pressure microfiltration assay.

CAMLs and the noted CAML associated structures may be used independently as cancer markers, or in combination with biomarker expression by other circulating cells, such as circulating tumor cells (CTCs). Suitable CTC subtypes include, but are not limited to, pathologically definable CTCs (PDCTCs), apoptotic CTCs, and the CTC subtype undergoing epithelial mesenchymal transition cells (EMTCTCs). Other suitable circulating cell types include circulating cancer associated vascular endothelial cells (CAVEs). The presence of these additional cell types themselves and also cell-free DNA (cfDNA), circulating tumor DNA (ctDNA), methylated DNA, proteomic, metabolomic, lipidomic and other biomarkers may provide a more complete understanding of a patient's disease.

When comparing the number of CAML associated structures in the circulating cells (e.g., CAMLs) between two subjects having cancer, it is preferable for the subjects to have the same type of cancer. However, it may be difficult to fully match two subjects in terms of the type of cancer, the stage of the cancer, the rate of progression of the cancer, history of treatment, and history of remission and/or reoccurrence of the cancer, among other factors. Therefore, it should be understood that there may be some variations in the characteristics in the cancers of two subject that are being compared in this method.

It should also be understood that the data to which the determinations are compared, e.g. the number of CAML associated structures from a subject having the same type of cancer, may be data from a single subject, or the combined results of data from two or more subjects. The predictive value of the methods of the invention will be increased over time as baselines are established on data from groups of subjects having the same or similar cancers. Therefore, as used herein, “a subject having the same type of cancer” means a single subject or data from a group of two or more subjects having the same type of cancer.

### Methods of Treatment

In each aspect and embodiment of the invention, the method optionally further comprises administering a therapeutically effective amount of a cancer treatment to the subject. The subject may be a subject in which the OS and PFS is predicted to be shorter than the OS and PFS of another subject.

The identity of the cancer treatment will correspond to the particular type of cancer being treated. However, suitable cancer treatments include chemotherapy, single drugs, combination of drugs, immunotherapy, radiation therapy, chemoradiation, chemoradiation combined with single or multiple drug, chemoradiation combined with single or multiple drugs (such as immunotherapy drugs), cell therapy, and other therapies.

Additional cancer treatments include, but are not limited to, immunotherapeutic agents, chemotherapeutic agents, radiotherapeutic agents, existing cancer drugs, CCR5 antagonists and CXCR4 antagonists. Examples of cancer treatments include, but are not limited to, one or more of antibodies or antagonists that block the activity of CCL3, CCL5 (RANTES), CCL7 or CCL8; Leronlimab (PRO 140); T-VEC, AM-0010, CXCR4 antagonist, TGF-beta kinase inhibitor galunisertib, anti-CSF-1R monoclonal antibody, Abemaciclib, Faslodex, necitumumab, AZD9291, Cyramza (ramucirumab), TPIV 200, Galunisertib, cancer vaccines, cytokines, cell-based therapies, bi- and multi-specific antibodies, tumor-targeting mAbs, Rituximab, oncolytic viruses, reovirus, Blinatumomab, Sipuleucel-T, T-Vec, IL-2, IFN-α, Trastuzumab, Celuximab, bevacizumab, Tim-3, BTLA, anti-IL-10, GM-CSF, anti-angiogenesis treatment, VEGF blockade, HMGB1, Nrp1, TAM receptor tyrosine kinases, Axl, MerTK, ALT-803, IL-15, Immunosuppressive Ligand Phosphatidylserine (PS), bavituximab, bevacizumab (anti-VEGF), coblmetinib (MEK inhibitor), vemurafenib (BRAF inhibitor), erlotinib (EGFR), alectinib (ALK inhibitor), bevacizumab (anti-VEGF), pazopanib (tyrosine kinase inhibitor), dabrafenib (BRAF inhibitor), trametinib (MEK inhibitor), durvalumab (anti-PD-L1), sunitinib (RTK inhibitor), pazopanib (RTK inhibitor), sargramostim, VISTA, TIM-3, LAG-3, PRS-343, CD137 (4-1BB)/HER2 bispecific, USP7, anti-HER2, SEMA4D, CTLA-4, PD-1, PD-L1, and PD-L2. As a non-limiting example, the treatment may be a cancer vaccine and the subject expresses at least one HLA allele. As a non-limiting example, the immunotherapy may be PD-L1 immunotherapy.

The subjects mentioned in the methods of the present invention will be a human, a non-human primate, bird, horse, cow, goat, sheep, a companion animal, such as a dog, cat or rodent, or other mammal. The subject having cancer may be undergoing treatment for the cancer. Such treatments include, but are not limited to targeted agents, chemotherapy, and radiation therapy.

### Companion Diagnostics

The information obtained using the methods of the invention can be used as a companion or complementary diagnostic. A companion or complementary diagnostic is a diagnostic test that can be used in combination with a therapeutic drug for a selected disease or condition. The companion diagnostic determines the suitability of the drug for treatment of the disease or condition in a particular patient, i.e. the companion diagnostic can help to predict whether the subject will be a responder or non-responder to the effects of the drug on the disease or condition in the subject. Thus, the companion diagnostic provides information that is used in treatment decisions and that is essential for the safe and effective use of a corresponding drug or biological product.

### EXAMPLES

### Sample Collection and Processing

Whole blood samples (7.5 mL) collected in CELLSAVE™ preservative tubes were processed with a CELLSIEVE™ Microfiltration Assay using a low-pressure vacuum system^{[1, 12]} or syringe pump^{[12]}. The CELLSIEVE™ Microfiltration Assay isolates circulating cells based on size exclusion, 7-8 micron diameter pores. A trained cytologist identified prognostically relevant CAMLs based on morphological features and the phenotypic expression of CD45, EpCAM, Cytokeratins 8, 18, 19 and DAPI^{[1, 6, 12]} using pre-established cytological features described^{[6, 11, 14]}. An Olympus BX54WI Fluorescent microscope with Carl Zeiss AxioCam and Zen2011 Blue (Carl Zeiss) was used for all imaging.

### Micronuclei

### Experiment 1

Micronuclei (MN) are a result of biological DNA repair mechanisms forming due to internal chromosomal aberrations which indicate sub-clonal cancer populations with higher cell survivability and drug therapy resistance. MN are often observed as small fragments of nucleic acids excised from a primary nucleus in circulating stomal cells (CStCs) as result of DNA damage^{1,2} (FIGS. 2 and 3). CStCs with damaged DNA undergoing repair mechanisms, such as those that form MN, appear to have upregulated expression of programmed cell death ligand (PD-L1). CAMLs were evaluated in metastatic breast cancer (mBC) patients for presence of MN and the cell's PD-L1 expression, to determine its prognostic significance to clinical outcomes.

MN formations were enumerated in CAMLs in a prospective pilot study using n=76 metastatic breast cancer (mBC) patients starting new lines of treatment. Whole peripheral blood (7.5 mL) was procured and filtered for CAMLs and then stained for PD-L1¹⁴. DAPI was used to identify MN, small (<3 μm) DAPI+ circular formations within the cytoplasm, separate from the primary nucleus. The number of MN was compared to PD-L1 expression in all CAMLs, and MN presence was also compared to all available clinical variables. Patients' progression-free survival (PFS) and overall survival (OS) hazard ratios (HRs) were analyzed by censored univariate analysis based on RECIST v1.1 over two-years.

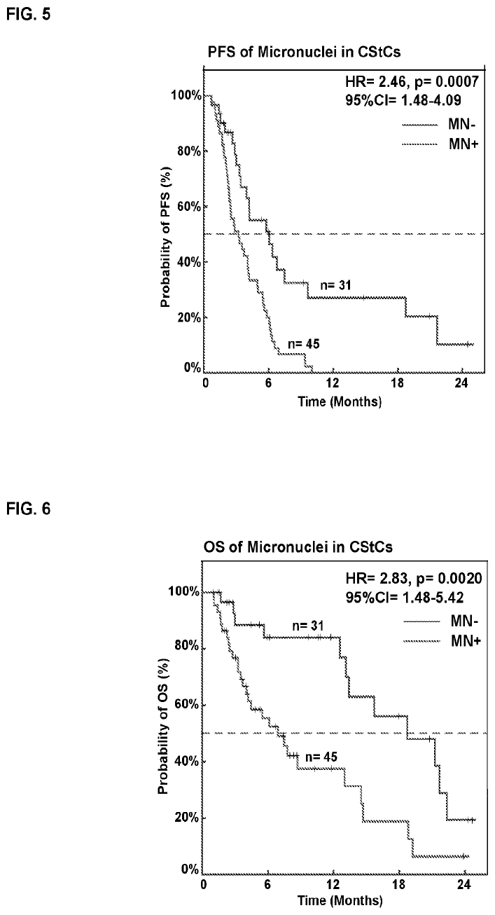

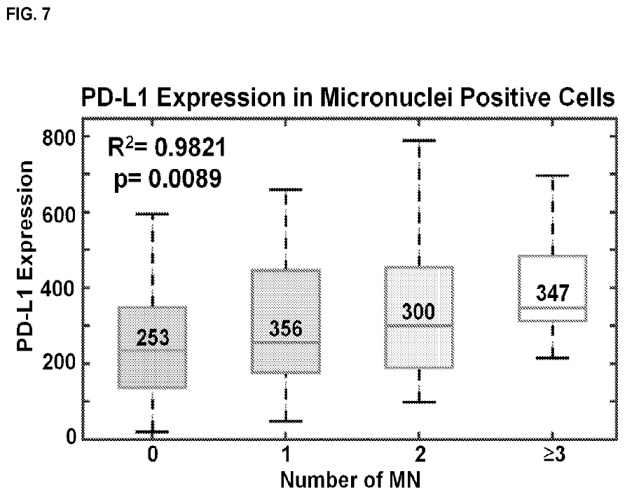

MN were identified in CAMLs in 59% (n=45/76) of patients. The presence of MN within CAMLs was significantly prognostic for worse PFS and worse OS over 24 months (FIGS. 5 and 6). MN positive CAMLs had a significantly higher PD-L1 expression than MN negative CAMLs (p=0.0082) (FIG. 7). Regression analysis identified a significant linear relationship between MN number and PD-L1 expression within CAMLs (R²=0.9821, p=0.0089).

### Experiment 2

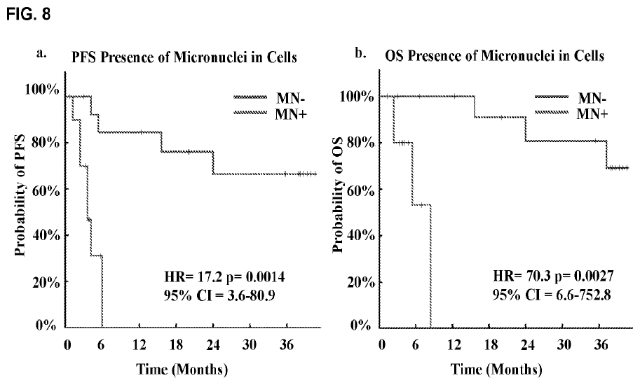

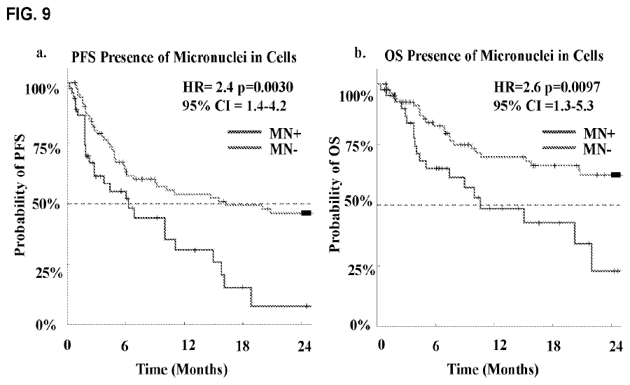

While MN formations in CAMLs from colorectal (n=25), breast (n=97) and an array of other cancers are uncommon, they were still observed in CAMLs in 44% of all colorectal patients and 60% of all breast cancer patients evaluated. Further, CAML MN presence was independently prognostic for progression free survival (PFS) (HR=17.2, 95% CI 3.6-80.9, p=0.001) and overall survival (OS) (HR=70.3, 95% CI 6.6-752.8, p=0.002) in colorectal patients (FIG. 8) and for PFS (HR=2.3, 95% CI 14-3.9, p=0.0019) and OS (HR=2.0, 95% CI 1.1-3.5, p=0.0362) in breast cancer patients. As can be seen, the presence of MN was associated with worse outcome for PFS and OS in these subjects.

Further, in an analysis of n=130 patients with an array of cancer types, MN presence in CAMLS was found to be predictive of worse PFS (HR=2.4, 95% CI 1.4-1.2, p=0.0030) and OS (HR=2.6, 95% CI 1.3-5.3, p=0.0097) (FIG. 9).

### Experiment 3

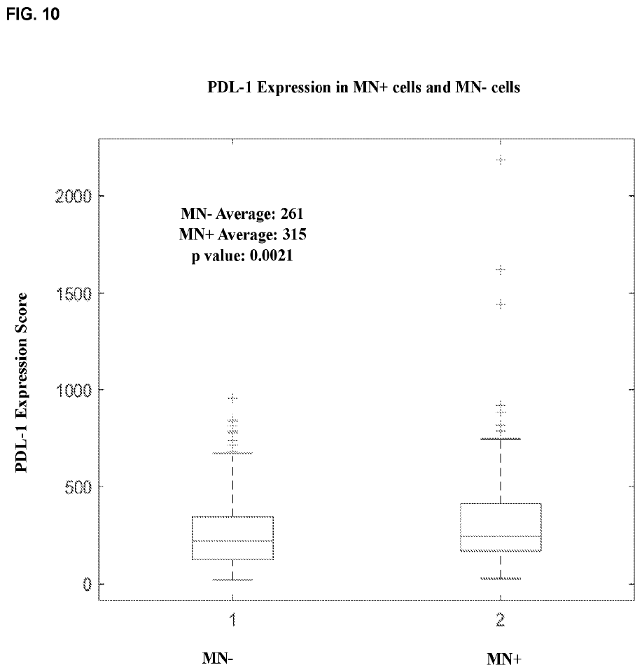

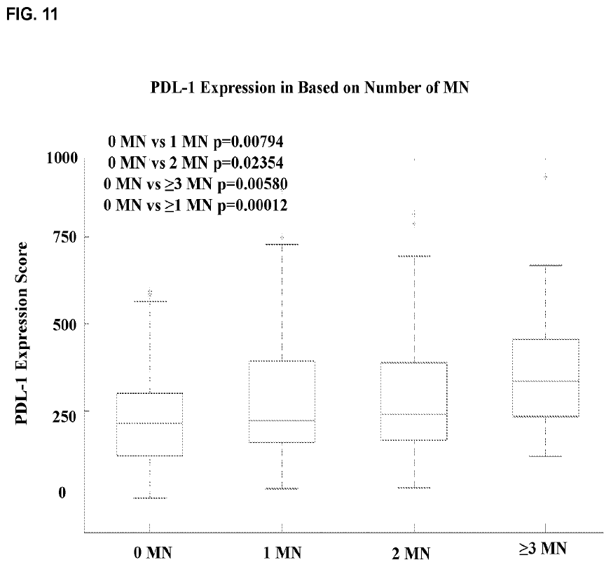

Additional assays were conducted to evaluated MN and PD-L1 co-expression in CAMLs of breast cancer patients. It was found that CAMLs with higher MN number had statistically higher PD-L1 expression (FIGS. 10 and 11) and also MN presence was correlated with better response to PD-L1/PD-1 immunotherapy induction (data not shown).

### Experiment 4

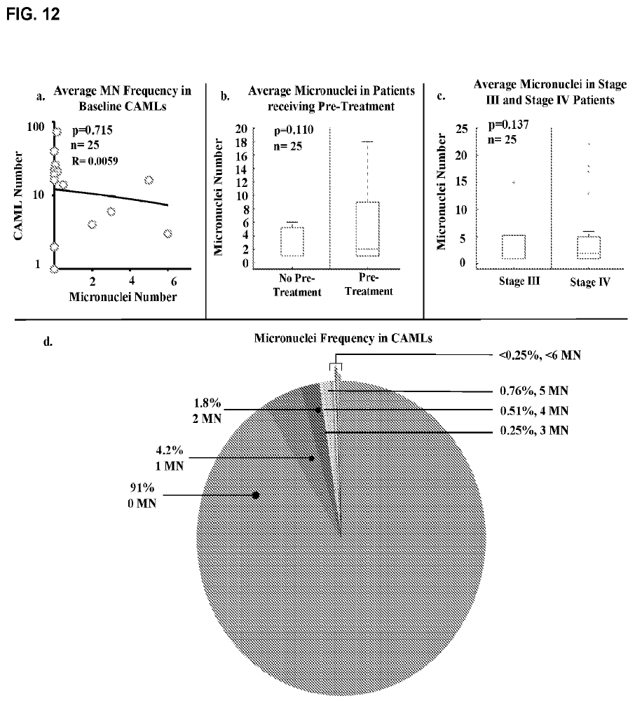

For patients with sequential samples from baseline before cancer therapy and following cancer therapy, change in the number of CAML MN can provide informative patient information. Adenocarcinoma patients (n=25) were treated with FOLFOX or FOLFIRI (Table 1). FIG. 12 provides the information of CAML MN analyzed for various parameters. FIG. 12a is linear regression plot of the relationship of CAML number versus MN number in each patient at baseline sample. FIG. 12b plots the number of MN for patients currently on chemotherapy (i.e. genotoxin) or patients that were newly diagnosed and treatment naive. FIG. 12c presents data on the average number of MN associated with non-metastatic (Stage III) or metastatic spread (Stage IV). FIG. 12d presents data of frequency of MN found in all CAMLs (n=776). 91% of CAMLs (blue) were negative for micronuclei, while only 9% were positive for at least 1 MN.

TABLE 1
Table 1. Clinical Demographic for patients

| Patient Demographics | n = 25 | |
|---|---|---|
| Age (median) | 51 | |
| Age IQR, Range | 45-61, 27-92 | |
| Gender | | |
| Male | 17 | (68%) |
| Female | 8 | (32%) |
| Race | | |
| Caucasian | 13 | (52%) |
| African American | 1 | (4%) |
| Hispanic | 1 | (4%) |
| Asian | 1 | (4%) |
| Unknown | 9 | (36%) |
| Stage | | |
| Non-metastatic (II-III) | 8 | (32%) |
| Metastatic (IV) | 17 | (68%) |
| Prior Treatment | | |
| Pre-Treatment | 13 | (52%) |
| No Pre-treatment | 12 | (48%) |
| Current Treatment | | |
| FOLFOX | 17 | (68%) |
| FOLFIRI | 4 | (16%) |
| Other** | 4 | (28%) |
| Histology | | |
| Adenocarcinoma* | 25 | (100%) |

*1 patient: identified as adenocarcinoma but diagnosed with appendix cancer
**Single agent inhibitor (i.e. cetuximab or Leronlimab)

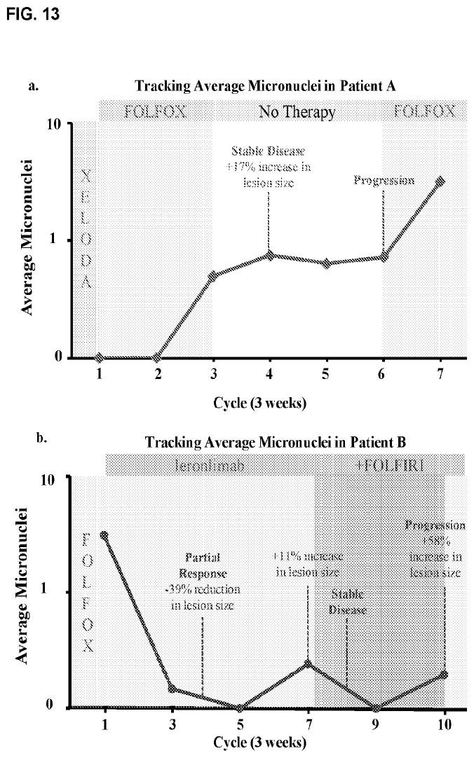

FIG. 13 presents two case studies that track CAML MN in blood draws. FIG. 13a corresponds to Patient A. After progression on maintenance therapy (Xeloda, red box), FOLFOX (orange box) was started. The number of CAML MN were found to increase after 4 therapy cycles which correlated to an increase in tumor (+17%). Therapy was halted due to an infection. The number of CAML MN then increased further correlating with a finding of new lesions at cycle 6. FOLFOX was then restarted, and the number of CAML MN continued to increase, the patient then dropped from study. FIG. 13b corresponds to Patient B, a patient with progressive disease on FOLFOX that was started on a single agent CCR5 inhibitor (Leronlimab, blue box), which was followed by a decrease in the number of CAML MN correlating with a reduction (−39%) in tumor size. The number of CAML MN then increased slightly, which correlated with a slight increase in tumor size (+11%) at which point FOLFIRI (purple box) was added to leronlimab. After FOLFIRI induction, a drop in the number of CAML MN was seen which correlated with stable disease.

### Extracellular Vesicles

### Experiment 1

Budding of extracellular structures on CAMLs has been observed in metastatic non-small cell lung carcinoma (mNSCLC) patients. In a prospective analysis of n=40 mNSCLC samples, extracellular vesicle (EV) budding was enumerated on CAMLs to determine if their formation had an effect on clinical outcomes. These preliminary results suggest that EV budding from CAMLs prognosticates for worse clinical outcome which may serve as the mechanism for cancer EV formation and spread throughout the body.

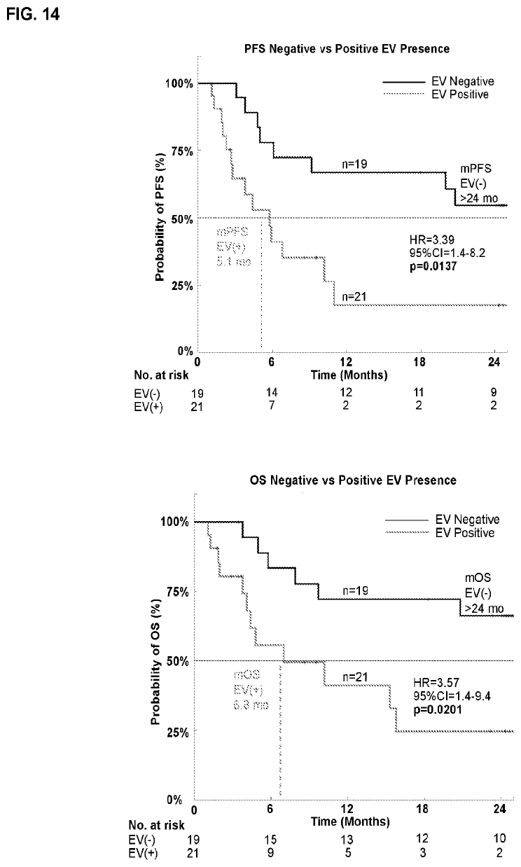

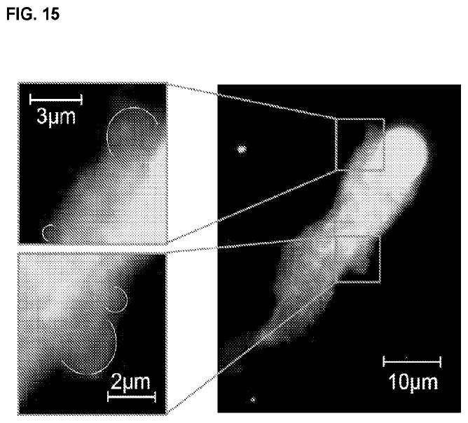

A single blind prospective pilot study was initiated to evaluate extracellular budding on the CAMLs of mNSCLC patients from blood samples obtained prior to therapy to determine their prevalence and clinical utility. Anonymized blood was procured and filtered to isolate CAMLs and stained for cytokeratin, CD45, CD31 and PD-L1. EV budding was observed as small (≤1 μm) bulbous protrusions from the cell periphery. EVs were quantified and compared against patient progression free survival (PFS) and overall survival (OS) with hazard ratios (HRs) at 24 months by censored univariate analysis. The imaged EVs were also characterized by their PD-L1 biomarker expression.
CAMLs were identified in 88% (n=35/40) of all samples
EV budding was identified in 60% (n=21/35) of CAMLs
Presence of EV budding in CAMLs was associated with significantly worse PFS (HR=3.39, 95% CI=1.4-8.2, p=0.0137) (FIG. 14, top panel)
Presence of EV budding in CAMLs was associated with significantly worse OS (HR=3.57, 95% CI=1.4-9.4, p=0.0201) (FIG. 14, bottom panel)
EVs were found to have positive PD-L1 expression in 57% (n=12/21) of CAMLs (FIG. 15)

EV budding found on phagocytic stromal cells in the blood appear with tumor positive biomarkers, predict faster disease progression and poor survival. These findings suggest that CAMLs are an origin cell for some cancer EVs and can be evaluated through liquid biopsy analysis.

### Experiment 2

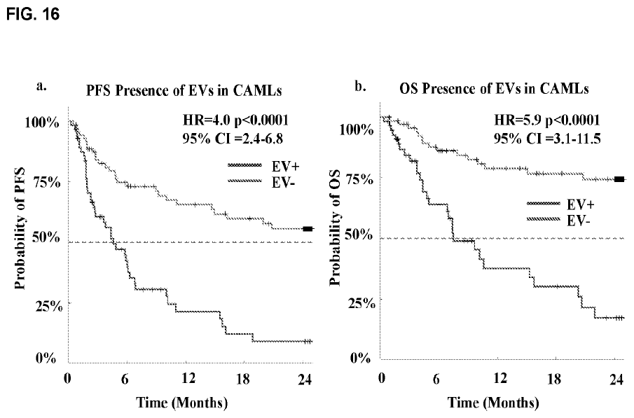

In an analysis of n=130 patients with an array of cancer types (breast n=29, colorectal n=25, esophageal n=1, lung n=43, ovarian n=1, pancreatic n=19, prostate n=2, sarcoma n=10), CAML EV presence was found to be predictive of worse PFS (HR=4.0, 95% CI 2.4-6.8, p<0.0001) and OS (HR=5.9, 95% CI 3.1-11.5, p<0.0001) (FIG. 16).

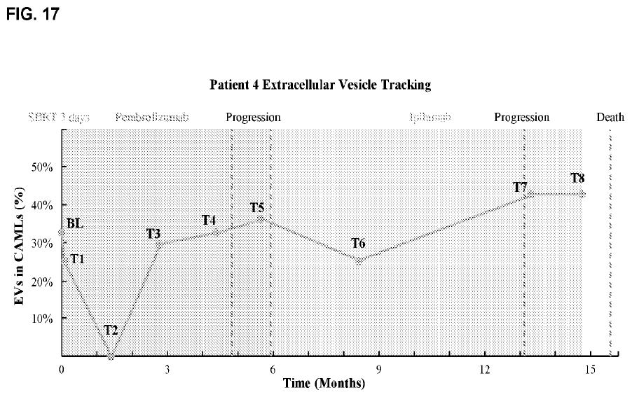

FIG. 17 tracks the percentage of CAMLs with EVs in a single patient undergoing chemoradiation therapy (CRT) over 15 months. Patient received CRT at baseline (BL) and CAML EV presence dropped 33% after CRT completion seen at the T1 blood draw. CAML EV presence increases from T2 to T5 at which point the patient was found to have disease progression as confirmed by radiographic imaging (PET/CT). The patient was started on Ipilumab and was then found to progressed with additional metastases at T7.

### Experiment 3

In a prospective analysis of mNSCLC patients (n=104), EV budding was enumerated on CAMLs to determine their clinical significance on Progression Free Survival (PFS)& Overall Survival (OS), further subtyping based on treatment with or without PD-L1 immunotherapy (IMT) based on standard of care treatment. These preliminary data suggests that EV positive (EV+) CAMLs prognosticates for worse outcomes in mNSCLC.

The single blind multi-year prospective study was initiated to investigate the relationship between EV budding in CAMLs to PFS & OS prior to start of new treatment lines for mNSCLC. Anonymized blood (7.5 mL) was procured from n=104 pathologically confirmed mNSCLC patients and filtered to isolate CAMLs to measure EV budding using tumor/EV markers (i.e. cytokeratin, CD163 or CD31) and immune specific marker (PD-L1). Blood was filtered by CELLSIEVE™ microfiltration and CAML EV budding characterized as small (<5 μm) bulbous protrusions from the cell cytoplasm. CAML EVs were quantified by presence (EV+) or absence (EV−) to compare PFS and OS with hazard ratios (HRs) at 60 months by censored univariate and multivariate analyses.

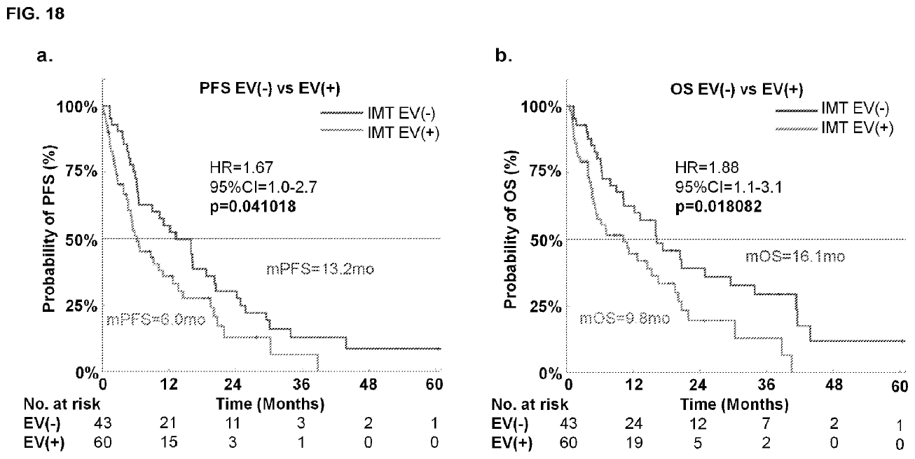

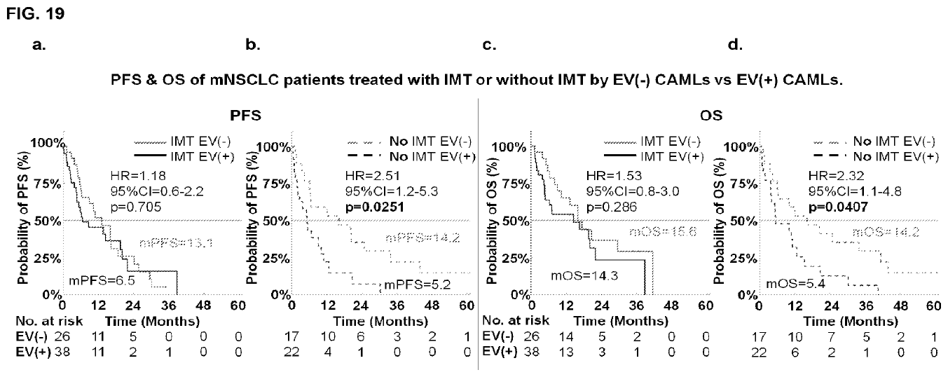

CAMLs were identified in 93% (n=97/104) of all samples. EV budding was identified in 62% (n=60/97) of samples with CAMLs. EV(+) CAMLs were associated with significantly worse PFS (HR=1.67, p=0.0410) and OS (HR=1.88, p=0.0180) (FIG. 18, panel a. and b. respectively). EV(+) patients NOT treated with immunotherapy (IMT) had significantly worse PFS (HR=2.51, p=0.0251) and OS (HR=2.32, p=0.0407) (FIG. 19). EV(+) patients appeared to benefit from additional IMT with longer mPFS and mOS (FIG. 19).

The results demonstrate that EV budding on phagocytic stromal cells found in the blood of mNSCLC patients predicts for poorer PFS and OS (FIG. 18 and Table 2). Poorer PFS and OS caused by EV presence in CAMLs is reduced with the addition of PD-L1 immunotherapy (FIG. 19).

TABLE 2
Patient demographics

| Median Age (Range) | | 66 | (42-82) |
|---|---|---|---|
| Sex (M/F) | | 63 (61%)/40 (39%) | |
| Race | White | 79 | (77%) |
| | Black | 18 | (17%) |
| | Other | 6 | (6%) |
| Histology | Adeno | 59 | (58%) |
| | NSCLC/Unknown | 23 | (22%) |
| | Squamous | 21 | (20%) |
| Smoker History | Never | 16 | (16%) |
| | Light (<50pks/yr) | 47 | (46%) |
| | Heavy (≥50pks/yr) | 33 | (32%) |
| | Unknown | 7 | (6%) |
| ECOG | 0 | 48 | (47%) |
| | ≥1 | 47 | (46%) |
| | Unknown | 8 | (7%) |
| Recurrence | Localized | 29 | (28%) |
| | Distant | 74 | (72%) |
| Immunotherapy | Pembro | 25 | (24%) |
| | Durva | 12 | (12%) |
| | Atezo | 11 | (11%) |
| | Other | 18 | (17%) |
| EV Presence | EV(−) | 43 | (42%) |
| | EV(+) | 60 | (58%) |

### Enlarged Polynuclearization

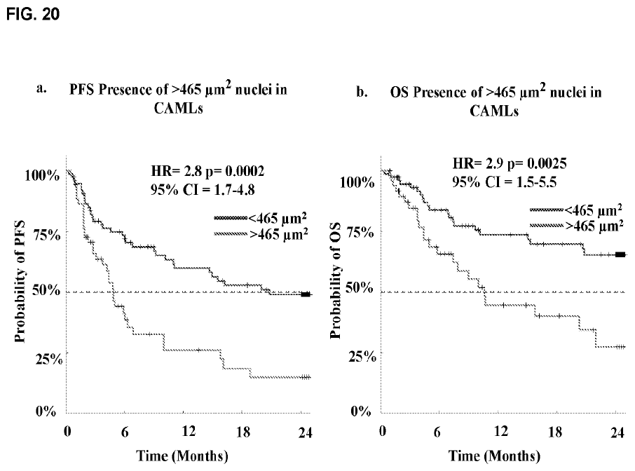

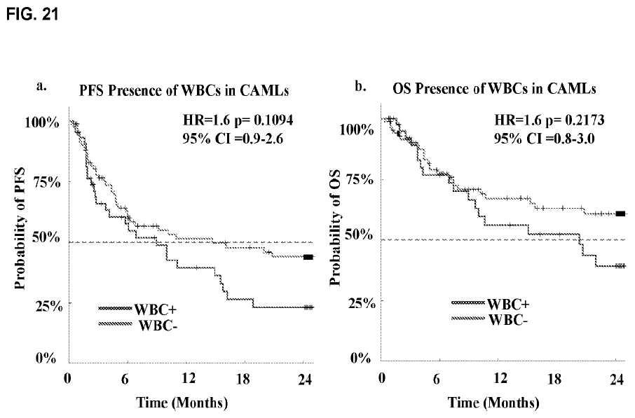

Enlarged polynuclearization (EPN) is a common criteria for identifying CAMLs, however the amount of nucleic acid enlargement and polynuclearization of the cell may be also be correlated with both cellular size and cellular aberration caused by cancer. A number of patients (n=130) with a variety of cancer types were evaluated and it was found that patients with larger nuclei masses, as measured by nuclear area, had more aggressive disease measured by PFS and OS. Patients with CAMLs with nuclear mass areas above of >465 μm² had worse PFS of (HR=2.8, 95% CI 1.7-4.8, p=0.000²) and OS (HR=2.9, 95% CI 1.5-5.5, p=0.0025) (FIG. 20).

### Internalization of Intact Cells

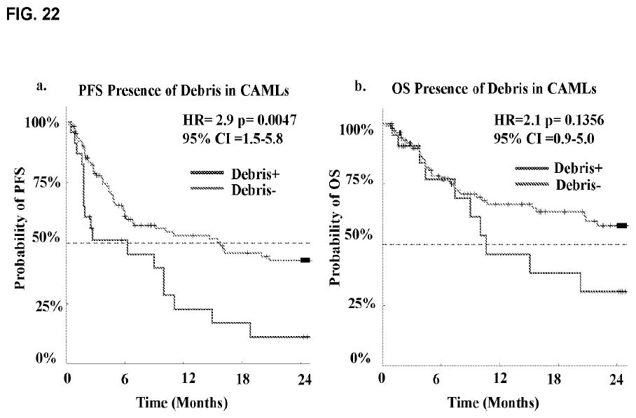

It has been previously shown that patients with internalized cells within CAMLs, e.g. CTCs, have worse clinical outcomes than without, though CAMLs can also be found with unusual internalized non-CTC cells, such as white blood cells, or other internal masses of unknown origin. However, this CAML internalization has been found to be more common in later stage diseased patients. CAMLs were evaluated in an array of cancer types, finding that CAMLs with internal whole or partial WBCs or internal whole or partial CTCs, or CAML bound WBCs, trended with worse clinical outcomes, including worse PFS (HR=1.6, 95% CI 0.9-2.6,p=0.1094) and OS (HR=1.6, 95% CI 0.8-3.0, p=0.2173) (FIG. 21). CAMLs were also evaluated in an array of cancer types and it was found that CAMLs with internal, non-disintegrated cellular debris or other internal structures were associated with worse clinical outcomes, including worse PFS (HR=2.9, 95% CI 1.5-5.8, p=0.0047) and OS (HR=2.1, 95% CI 0.9-5.0, p=0.1356) (FIG. 22).

### Combination of Properties

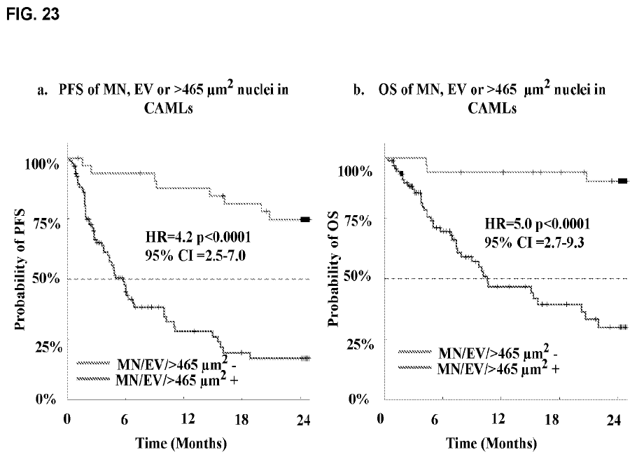

Based on the results of the experiments set forth above, combinations of the observable biological cellular characteristics described above were evaluated and various combination were found to allow stratification of patients with worse clinical outcomes, with the optimal combination in the n=130 set of multiple cancers being a combination of CAML MN, CAML EVs, and CAMLs with nuclei>465 μm², being predictive for worse PFS (HR=4.2, 95% CI 2.5-7.0, p<0.0001) and worse OS (HR=5.0, 95% CI 2.7-9.3, p<0.0001 (FIG. 23).

### Metastatic Disease

In additional analysis, it was further found that of the n=40 patients initially treated for non-metastatic disease, n=18 patients were re-diagnosed with metastatic disease within 2 years, with 72% having found metastatic disease within 10 months. In the n=22 patients not to be re-diagnosed with metastasis, 1 had CAML MN, 1 had CAML debris, 5 had CAML WBCs, 1 had a CAML EV. In the n=18 patients that were found to have progressed with metastasis within 2 years, 5 had CAML MN, 4 had CAML debris, 7 had CAML WBCs, 1 had a CAML bound CTC, and 13 had a CAML EV. This indicates that the presence of CAML EVs were 85% accurate at predicting patients that will rapidly relapse with metastatic disease. Further, the presence of CAML EVs or CAML MNs in patient blood was 90% accurate at predicting patients that will be quickly re-diagnosed with metastasis. Because samples were taken prior to therapy start, this suggests that CAMLs with MN or EVs may identify patients that were initially under-diagnosed, and likely had metastatic disease at time of blood draw.

### CITATIONS

1. Adams D L, Zhu P, Makarova O V, Martin S S, Charpentier M, Chumsri S, et al. The systematic study of circulating tumor cell isolation using lithographic microfilters. RSC Advances. 2014; 4:4334-42.
2. Lianidou E S, Markou A. Circulating tumor cells in breast cancer: detection systems, molecular characterization, and future challenges. Clinical chemistry. 2011; 57:1242-55.
3. Pantel K, Brakenhoff R H, Brandt B. Detection, clinical relevance and specific biological properties of disseminating tumour cells. Nature reviews Cancer. 2008; 8:329-40.
4. Paterlini-Brechot P, Benali N L. Circulating tumor cells (CTC) detection: clinical impact and future directions. Cancer letters. 2007; 253:180-204.
5. Adams D, Tsai S, Makarova O V, Zhu P, Li S, Amstutz P T, et al. Low cytokeratin- and low EpCAM-expressing circulating tumor cells in pancreatic cancer. ASCO Annual Meeting Proceedings; 2013. p. 11046.
6. Adams D L, Stefansson S, Haudenschild C, Martin S S, Charpentier M, Chumsri S, et al. Cytometric characterization of circulating tumor cells captured by microfiltration and their correlation to the cellsearch((R)) CTC test. Cytometry Part A: the journal of the International Society for Analytical Cytology. 2015; 87:137-144.
7. Krebs M G, Hou J M, Sloane R, Lancashire L, Priest L, Nonaka D, et al. Analysis of circulating tumor cells in patients with non-small cell lung cancer using epithelial marker-dependent and -independent approaches. J Thorac Oncol. 2012:7:306-15.
8. Farace F, Massard C, Vimond N, Drusch F, Jacques N, Billiot F, et al. A direct comparison of CellSearch and ISET for circulating tumour-cell detection in patients with metastatic carcinomas. British journal of cancer. 2011; 105:847-53.
9. Lecharpentier A, Vielh P, Perez-Moreno P, Planchard D, Soria J C, Farace F. Detection of circulating tumour cells with a hybrid (epithelial/mesenchymal) phenotype in patients with metastatic non-small cell lung cancer. British journal of cancer. 2011; 105:1338-41.
10. Adams D L, Adams D K, Stefansson S, Haudenschild C, Martin S S, Charpentier M, et al. Mitosis in circulating tumor cells stratifies highly aggressive breast carcinomas. Breast cancer research: BCR. 2016; 18:44.
11. Adams D L, Adams D K, Alpaugh R K, Cristofanilli M, Martin S S, Chumsri S, et al. Circulating Cancer-Associated Macrophage-Like Cells Differentiate Malignant Breast Cancer and Benign Breast Conditions. Cancer Epidemiol Biomarkers Prev. 2016; 25:1037-42.
12. Adams D L, Alpaugh R K, Martin S S, Charpentier M, Chumsri S, Cristofanilli M, et al. Precision microfilters as an all in one system for multiplex analysis of circulating tumor cells. RSC Advances. 2016; 6:6405-14.
13. Adams D L, Alpaugh R K, Tsai S, Tang C M, Stefansson S. Multi-Phenotypic subtyping of circulating tumor cells using sequential fluorescent quenching and restaining. Sci Rep. 2016; 6:33488.
14. Adams D L, Martin S S, Alpaugh R K, Charpentier M, Tsai S, Bergan R C, et al. Circulating giant macrophages as a potential biomarker of solid tumors. Proceedings of the National Academy of Sciences of the United States of America. 2014; 111:3514-9.
15. Allard W J, Matera J, Miller M C, Repollet M, Connelly M C, Rao C, et al. Tumor cells circulate in the peripheral blood of all major carcinomas but not in healthy subjects or patients with nonmalignant diseases. Clin Cancer Res 2004; 10:6897-904.
16. Anantharaman A, Friedlander T, Lu D, Krupa R, Premasekharan G, Hough J, et al. Programmed death-ligand 1 (PD-L1) characterization of circulating tumor cells (CTCs) in muscle invasive and metastatic bladder cancer patients. BMC Cancer. 2016; 16:744.
17. Mu Z, Benali-Furet N, Uzan G, Znaty A, Ye Z, Paolillo C, et al. Detection and Characterization of Circulating Tumor Associated Cells in Metastatic Breast Cancer. Int J Mol Sci. 2016; 17.
18. Lin S H, He J, Edelman M, Xu T, Gao H, Reuben J, et al. Sequential Assessment of DNA Damage Response and PD-L1 Expression in Circulating Tumor Cells of Lung Cancer Patients during Radiotherapy. JOURNAL OF THORACIC ONCOLOGY; 2015: ELSEVIER SCIENCE INC 360 PARK AVE SOUTH, NEW YORK, NY 10010-1710 USA. p. S266-S7.
19. Zhu P, Stanton M L, Castle E P, Joseph R W, Adams D L, Li S, et al. Detection of tumor-associated cells in cryopreserved peripheral blood mononuclear cell samples for retrospective analysis. J Transl Med. 2016; 14:198.
20. Daniel L. Adams. R. Katherine Alpaugh, Steven H. Lin, Jeffrey R. Marks, Raymond Bergan, Stuart S. Martin, Saranya Chumsri, Massimo Cristofanilli, Cha-Mei Tang, Steingrimur Stefansson, “Multiplex phenotyping of circulating cancer associated macrophage-like cells in patients with solid tumors”, Proceedings of AACR, Vol. 58, April 2017. Abstract #778.
21. International Patent Application Publication No. WO 2013/181532, dated Dec. 5, 2013.
22. International Patent Application Publication No. WO 2016/33103, dated Mar. 3, 2016.
23. Jiao et al., CCR5 Governs DNA Damage Repair and Breast Cancer Stem Cell Expansion. Cancer Res. 78(7):1657-1671 (2018).
24. Vangelista, L. and S. Vento. The Expanding Therapeutic Perspective of CCR5 Blockade. Frontiers in Immunology 8: Article 1981 (2018).
25. Singh et al. CCR5/CCL5 axis interaction promotes migratory and invasiveness of pancreatic cancer cells. Nature Scientific Reports 8:1323, 12 pages (2018).
26. Velasco-Velazquez et al. The potential to target CCL5/CCR5 in breast cancer. Expert Opin. Ther. Targets 18(11):1265-1275 (2014).
27. Cooper G M. The Cell: A Molecular Approach. 2nd edition. Sunderland (MA): Sinauer Associates (2000). Endocytosis.
28. Oppermann M. Chemokine receptor CCR5: insights into structure, function, and regulation. Cell Signal. 16:1201-1210 (2004).
29. Signoret, N., Christophe, T., Oppermann, M., and Marsh, M. pH-independent endocytic cycling of the chemokine receptor CCR5. Traffic 5, 529-543 (2004).
30. Berro, R. et al. Multiple CCR5 conformations on the cell surface are used differentially by human immunodeficiency viruses resistant or sensitive to CCR5 inhibitors. J. Virol. 85, 8227-8240 (2011).
31. Fox J M, Kasprowicz R, Hartley O, Signoret N. CCR5 susceptibility to ligand-mediated down-modulation differs between human T lymphocytes and myeloid cells. J Leukoc Biol. 98:59-71 (2015).
32. Khalid A, Wolfram J, Ferrari I, Mu C, Mai J, Yang Z, Zhao Y, Ferrari M, Ma X, Shen H. Recent advances in discovering the role of CCL5 in metastatic breast cancer. Mini Rev Med Chem. 15:1063-1072 (2015).
33. Kasabwala D M, Bergan R C, Gardner K P, Lapidus R, Tsai S, Aldakkak M, Adams D L. Micronuclei in Circulating Tumor Associated Macrophages Predicts Progression in Advanced Colorectal Cancer. Biomedicines 2022, 10, 2898.
34. Fenech M, Kirsch-Volders M, Natarajan A T, Surralles J. Crott J W, Parry J, Norppa H, Eastmond D A, Tucker J D, Thomas P. Molecular mechanisms of micronucleus, nucleoplasmic bridge and nuclear bud formation in mammalian and human cells. Mutagenesis 2011, 26, 125-132.

1. A method for predicting overall survival (OS) and/or progression free survival (PFS) of a subject having cancer, said method comprising determining the presence of one or more of (a) micronuclei (MN), (b) extracellular vesicles (EVs), (c) enlarged polynuclearization (EPN), (d) one or more internalized intact cells, and (e) internal cellular debris in circulating cells or produced by circulating cells of a biological sample from a subject having cancer, wherein the presence of one or more of (a), (b), (c), (d) and (e) predicts lower OS and/or PFS than a subject having the same cancer without the presence of one or more of (a), (b), (c), (d) or (e).

2. A method for predicting presence of metastatic spread and/or metastatic progression in a subject having cancer, said method comprising determining the presence of one or more of (a) MN, (b) EVs, (c) EPN, (d) internalized intact cells, and (e) internal cellular debris in circulating cells or produced by circulating cells of a biological sample from a subject having cancer, wherein the presence of one or more of (a), (b), (c), (d) and (e) predicts presence of metastatic spread and/or metastatic progression in the subject.

3. A method for predicting cancer progression in a subject having cancer comprising determining the presence of one or more of (a) MN, (b) EV, (c) EPN, (d) internalized intact cells, and (e) internal cellular debris in circulating cells or produced by circulating cells in a first biological sample and a second biological sample at a later date, and optional additional biological samples, obtained from a subject having cancer, wherein when one or more of (a), (b), (c), (d) or (e) is present in or produced by the circulating cells of the second and/or additional biological sample but not present in or produced by the circulating cells of the first biological sample, the cancer is predicted to progress in the subject, and wherein when one or more of (a), (b), (c), (d) or (e) is present in or produced by the circulating cells of the first biological sample but not present in or produced by the circulating cells of the second and/or additional biological sample, the cancer is predicted not to progress in the subject.

4. A method for predicting cancer progression in a subject having cancer comprising determining the presence of one or more of (a) MN, (b) EV, (c) EPN, (d) internalized intact cells, and (e) internal cellular debris in circulating cells or produced by circulating cells in a first biological sample and a second biological sample at a later date, and optional additional biological samples, obtained from a subject having cancer, wherein the first sample is obtained from the subject prior to or during cancer treatment, wherein the second sample and optional additional samples are obtained from the subject after at least one cancer treatment, wherein when one or more of (a), (b), (c), (d) or (e) is present in or produced by the circulating cells of the first biological sample but not present in or produced by the circulating cells of the second and/or additional biological sample, the cancer is predicted not to progress, and wherein when one or more of (a), (b), (c), (d) or (e) is present in the second and optional/or additional samples but not present in or produced by the circulating cells of the first biological sample, the cancer is predicted to progress.

5. A method for predicting response to treatment in a subject having cancer comprising determining the presence of one or more of (a) MN, (b) EV, (c) EPN, (d) internalized intact cells, and (e) internal cellular debris in circulating cells or produced by circulating cells in a first biological sample and a second biological sample at a later date, and optional additional biological samples, obtained from a subject having cancer, wherein the first sample is obtained from the subject prior to or during cancer treatment, wherein the second sample and optional additional samples are obtained from the subject after at least one cancer treatment, wherein when one or more of (a), (b), (c), (d) or (e) is present in or produced by the circulating cells of the first biological sample but not present in or produced by the circulating cells of the second and/or additional biological sample, the subject is predicted to respond to the treatment, and wherein when one or more of (a), (b), (c), (d) or (e) is present in the second and optional/or additional samples but not presence in the circulating cells of the first biological sample, the subject is predicted not to respond to the treatment.

6. The method of claim 1, wherein said OS and/or PFS is over a period of at least 12 months or wherein said OS and/or PFS is over a period of at least 24 months.

7. (canceled)

8. The method of claim 1, wherein the size of the biological sample is between 5 and 15 mL.

9. The method of claim 1, wherein the circulating cells have the following characteristics:
(a) multiple individual nuclei and/or one or more fused nuclei having a size of about 14-64 μm;
(b) cell size of about 20-300 μm in size; and
(c) morphological shape selected from the group consisting of spindle, tadpole, round, oblong, two legs, more than two legs, thin legs, and amorphous.

10. The method of claim 9, wherein the circulating cells have one or more of the following additional characteristics:
(d) CD14 expression;
(e) CD45 expression;
(f) EpCAM expression;
(g) vimentin expression;
(h) PD-L1 expression;
(i) monocytic and macrophage CD11c marker expression;
(j) endothelial CD146 marker expression;
(k) endothelial and macrophage CD202b marker expression;
(l) endothelial, macrophage and white blood cell CD31 marker expression; and
(m) epithelial cancer cell CK8, 18, and/or 19 marker expression.

11. The method of claim 1, wherein the source of the biological sample is one or more of peripheral blood, blood, lymph node, bone marrow, cerebral spinal fluid, and urine.

12. The method of claim 11, wherein the biological sample is antecubital-vein blood, inferior-vena-cava blood, femoral vein blood, portal vein blood, or jugular-vein blood.

13. The method of claim 1, wherein the cancer is a Stage I cancer, Stage II cancer, Stage III cancer, Stage IV cancer, carcinoma, sarcoma, neuroblastoma, melanoma, epithelial cell cancer, lung cancer, breast cancer, prostate cancer, pancreatic cancer, bladder cancer, kidney cancer, head and neck cancer, colorectal cancer, liver cancer, ovarian cancer, osteosarcoma, esophageal, brain & ONS, larynx, bronchus, oral cavity and pharynx, stomach, testis, thyroid, uterine cervix, uterine corpus cancer or other solid tumor cancers.

14. The method of claim 1, wherein circulating cells are isolated from the biological samples for the determining steps using one or more means selected from the group consisting of size exclusion methodology, immunocapture, red blood cell lysis, white blood cell depletion, a high-molecular weight polysaccharide such as FICOLL®, electrophoresis, dielectrophoresis, flow cytometry, magnetic levitation, and various microfluidic chips, slits, channels, hydrodynamic size-based sorting, grouping, trapping, concentrating large cells, eliminating small cells, or a combination thereof.

15. The method of claim 14, wherein circulating cells are isolated from the biological samples using size exclusion methodology that comprises using a microfilter.

16. The method of claim 15, wherein the microfilter has a pore size ranging from about 5 microns to about 20 microns.

17. The method of claim 16, wherein the pores of the microfilter have a round, race-track shape, oval, square and rectangular pore shape.

18. The method of claim 16, wherein the microfilter has precision pore geometry and uniform pore distribution.

19. The method of claim 14, wherein circulating cells are isolated using a microfluidic chip via physical size-based sorting, hydrodynamic size-based sorting, grouping, trapping, immunocapture, concentrating large cells, or eliminating small cells based on size.

20. The method of claim 1, wherein circulating cells are isolated from the biological samples for the determining steps using a microfiltration assay.

21. The method of claim 4, wherein the treatment is one or more of chemotherapy, single drug, combination of drugs, immunotherapy, radiation therapy, chemoradiation, radiation combined with single or multiple drugs, chemoradiation combined with single or multiple drugs, cancer vaccine, and cell therapy.

22. The method of claim 21, wherein the treatment is a cancer vaccine and the subject expresses at least one HLA allele.

23. The method of claim 1, wherein the subject is being treated with one or more of chemotherapy, single drug, combination of drugs, immunotherapy, radiation therapy, chemoradiation, radiation combined with single or multiple drugs, chemoradiation combined with single or multiple drugs, cancer vaccine, and cell therapy.

24. The method of claim 23, wherein the immunotherapy is PD-L1 immunotherapy.
